(19) United States
(12) Patent Application Publication
(10) Pub. No.: US 2024/0306889 A1
(43) Pub. Date: Sep. 19, 2024

(54) SCOPE SYSTEM AND METHODS FOR USING SAME

(71) Applicant: Sinus Clear Innovations, LLC, Gulfport, MS (US)

(72) Inventors: Vincent Pisciotta, Gulfport, MS (US); Clayton Bratton, Gulfport, MS (US)

(21) Appl. No.: 18/607,419

(22) Filed: Mar. 15, 2024

Related U.S. Application Data
(60) Provisional application No. 63/490,523, filed on Mar. 15, 2023.
(60) Provisional application No. 63/587,722, filed on Oct. 3, 2023.

(51) Int. Cl. A61B 1/00; A61B 1/015

(52) U.S. Cl. CPC A61B 1/00068; A61B 1/00096; A61B 1/00105; A61B 1/00126; A61B 1/00128; A61B 1/00135; A61B 1/015

(57) ABSTRACT

Provided herein are scope devices, systems and methods for using a disclosed scope device for an endoscopic procedure. In one aspect, a disclosed device may include a hand piece with a suction and irrigation hand piece which accommodates a sinus scope which allows the scope lens to be cleaned without removing the scope from the operative field.

### RELATED APPLICATION

This application claims the benefit of priority to U.S. patent application Ser. No. 63/490,523, filed Mar. 15, 2023, and U.S. patent application Ser. No. 63/587,722, filed Oct. 3, 2023, which are hereby incorporated in their entireties.

### BACKGROUND

Doctors often use endoscopes to see inside the body of a patient for performing surgical procedures. For example, endoscopes are often used in the ears, noses, and throats of patients to observe an operative field space during a medical procedure.

Endoscopes have a lens that may become dirty or fouled by blood or other fluids. During a medical procedure, if the lens is fouled such that the user cannot see through the endoscope, the user removes the endoscope from the patient to clean the lens. While not hearing a clear view of the operative field because of debris on the operative scope creates additional risk of the procedure, removing the endoscope from the patient for cleaning is undesirable as it interrupts the flow of the procedure and increases the time of the procedure among other things.

During a medical procedure the operation area may become filled with blood or other fluids. To remove fluids from the operation area, users use a suction device. The use of the suction device in some procedures may be performed by removing the endoscope and introducing the suction device into the body of the patient. When a user completes such a procedure, the user may remove the suction device and introduce the endoscope. Removing the endoscope to perform a suction procedure is undesirable because it increases the time for the procedure.

Many endoscopes do not provide comfortable hand grips. Rather, many endoscopes have little area for a user to comfortably grip and retain an endoscope.

Some endoscopes include suction or irrigation features, however, the controls for the suction and irrigation features are electromechanical. Electromechanical control devices tend to not provide a steady stream of irrigation fluid or a suction vacuum and may be costly.

Accordingly, there remains a need for an improved endoscope device and system to address the above-mentioned shortcomings. Furthermore, it would be desirable for the improved endoscope to have a constant and continuous stream of irrigation fluid or suction from a vacuum, which improves the operation of the system. A suction system that has a comfortable grip area to allow the user to operate the endoscope without discomfort would also be desirable. It would also be desirable for an endoscope system to have features that allow the user to perform irrigation and provide suction to an operation area while providing a constant flow of irrigation fluid and a constant vacuum during operations. These needs and other needs are met by the various aspects of the present disclosure.

### BRIEF OVERVIEW

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

The present disclosure relates to scope apparatuses, devices, and systems for endoscopic procedures. In one aspect, the disclosure relates to an endoscopic apparatus comprising: a body portion having an inner cavity operative to engage and retain an endoscope; a first tubing portion having a first distal end and a second distal end arranged in the inner cavity; a fluid port operative to engage the first distal end of the first tubing portion; a flow assembly operative to engage the second distal end of the first tubing portion and a portion of the endoscope; and a hand grip portion arranged on the body portion.

In another aspect, the disclosure relates to a surgical hand piece apparatus for endoscopic procedures, comprising: a body portion configured to receive and retain an endoscope; an adjustable sheath coupled to the body portion, wherein the adjustable sheath is configured to accommodate endoscopes of varying lengths; a combined suction and irrigation port integrated into the body portion; a mechanical foot pedal assembly communicatively connected to the combined suction and irrigation port, the mechanical foot pedal assembly including spring-loaded pinch valves for controlling fluid flow; and an ergonomic hand grip portion formed on the body portion. The ergonomic hand grip portion may include a soft overmolded material designed to reduce carpal tunnel pressure and fatigue during use.

In another aspect, the disclosure relates to a surgical hand piece apparatus for endoscopic procedures, comprising: a body configured to accommodate an endoscope; an ergonomic grip portion designed to conform to the contours of a user's hand; an integrated adjustable sheath for encasing a portion of the endoscope and allowing length adjustment; a combined suction and irrigation port connected to the body for providing fluid flow and suction through the endoscope; and a thenar rest positioned to support the hand piece on the user's thenar eminence during use.

In another aspect, the disclosure relates to a modular surgical hand piece apparatus for endoscopic procedures, comprising: a main body with a universal interface for attaching various ergonomic grip modules; a selection of interchangeable ergonomic grip modules designed to accommodate different hand sizes and preferences; a detachable adjustable sheath component for encasing an endoscope, with variable length settings; a set of interchangeable fluid control modules for customizing irrigation and suction capabilities; and a quick-connect mechanism for attaching and detaching the ergonomic grip modules and fluid control modules to the main body.

In another aspects, the disclosure relates to an endoscopic apparatus with integrated fluid control. The endoscopic apparatus may include a surgical hand piece with an embedded fluid control system for managing irrigation and suction during endoscopic procedures; a dual-channel tubing system within the hand piece, one for delivering irrigation fluid to the operative field and another for suction removal of fluids. In further aspects, the endoscopic device may further include a valve assembly operable to independently control the flow rate of irrigation and suction; a fluid reservoir connectable to the irrigation channel for supplying the irrigation fluid; and a waste collection container connectable to the suction channel for collecting fluids removed from the operative field.

In another aspect, the disclosure relates to an endoscopic apparatus, comprising: a surgical hand piece with a contoured grip conforming to human hand ergonomics to reduce fatigue during prolonged use; a weight distribution system within the hand piece to balance the apparatus and minimize strain on the user's wrist and arm; a textured surface on the grip areas to enhance grip stability and prevent slippage; and an adjustable support features to accommodate various hand sizes and grip styles. The apparatus may further include a user interface positioned to allow intuitive access to controls without requiring significant hand movement.

Various embodiments may include an adjustable sheath. The adjustable sheath may include one or more of a sheath body configured to encase a portion of an endoscope; an adjustment mechanism integrated into the sheath body for altering the effective length of the sheath to accommodate endoscopes of varying lengths; a fluid channel within the sheath body designed to direct irrigation fluid towards the distal end of the endoscope and to facilitate suction from the proximal end; a securement feature for maintaining a fluid-tight connection between the sheath body and the endoscope; and/or an attachment interface for removably coupling the adjustable sheath apparatus to the surgical hand piece. Various embodiments may also include a mechanical foot pedal assembly for controlling suction and irrigation, wireless communication and connectivity; and/or image capture and transmission capabilities.

In further aspects, the disclosure relates to methods for making, operating, and using the disclosed scope devices and apparatuses.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1 illustrates a planar view of a scope system in accordance with an embodiment of the present invention.

FIG. 2 illustrates a side view of the system in accordance with an embodiment of the present invention.

FIG. 3 illustrates a top view of the system in accordance with an embodiment of the present invention.

FIG. 4 illustrates a bottom view of the system in accordance with an embodiment of the present invention.

FIG. 5 illustrates a front view of the system in accordance with an embodiment of the present invention.

FIG. 6 illustrates a rear view of the system in accordance with an embodiment of the present invention.

FIG. 7 illustrates a cut-away view along the line A-A (of FIG. 6) of the device in accordance with an embodiment of the present invention.

FIG. 8 illustrates a perspective view of a scope system in accordance with another embodiment of the present invention.

FIG. 9 illustrates another view of a scope system in accordance with another embodiment of the present invention.

FIG. 10 illustrates another view of a scope system in accordance with another embodiment of the present invention.

FIG. 11 illustrates another view of a scope system in accordance with another embodiment of the present invention.

FIG. 12 illustrates another view of a scope system in accordance with another embodiment of the present invention.

FIG. 13 illustrates another view of a scope system in accordance with another embodiment of the present invention.

FIG. 14 illustrates another view of a scope system in accordance with another embodiment of the present invention.

FIG. 15 illustrates another view of a scope system in accordance with another embodiment of the present invention.

FIG. 16 illustrates another view of a scope system in accordance with another embodiment of the present invention.

FIG. 17 illustrates another view of a scope system in accordance with another embodiment of the present invention.

FIG. 18 illustrates another view of a scope system in accordance with yet another embodiment of the present invention.

FIG. 19 illustrates another view of a scope system in accordance with yet another embodiment of the present invention.

FIGS. 20-22 illustrate various views of a scope system with a large handpiece in accordance with yet another embodiment of the present invention.

FIGS. 23-25 illustrate various views of a scope system with a small handpiece in accordance with yet another embodiment of the present invention.

FIG. 26 is a flow chart showing a method for using a disclosed scope system in accordance with an embodiment of the present disclosure.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term-differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

### Device and System Overview

The present disclosure includes many aspects and features. Moreover, while many aspects and features relate to, and are described in, the context of endoscope and associated procedures, embodiments of the present disclosure are not limited to use only in this context. It will be appreciated that an apparatus, device, and system (which may collectively be referred to herein as a “scope device” or “endoscopic apparatus”) according to the present disclosure may be suitable for use in any medical procedure that involves a scope, such as endoscopic procedures. In particular, apparatus, devices and systems according to the present disclosure may be useful in medical procedures that require scope cleaning during a procedure. To this end, physicians and practitioners use endoscopes to see inside the body of a patient during medical procedures. Endoscopes (scopes) are often used during procedures in the ear, nose, and throat areas, and may be used in a variety of other medical procedures such as minimally invasive surgeries and orthopedic procedures.

During a medical procedure, the user introduces the endoscope into the body of a patient. The endoscope includes an optic portion that includes a lens proximate to a distal end of the endoscope. During the procedure, the lens may become obscured by blood, tissue, or other fluids. To continue the procedure, the user removes the endoscope from the patient and cleans the lens. During the procedure, when the user desires to remove fluids around the endoscope, the user may remove the endoscope and insert a suction device. Though some endoscopes may include irrigation and suction features, these devices often include electromechanical control valves that are incapable of providing a substantially continuous flow of irrigation fluid and suction. Additionally, endoscopes often lack a comfortable hand grip to allow the user to use the device for long periods of time.

In one aspect, disclosed herein is a hand piece with a suction and irrigation hand piece which accommodates a sinus scope which allows the scope lens to be cleaned without removing the scope from the operative field. In further aspects, it can allow irrigation with an antibiotic solution, warm water, or saline solution allowing for clearing of the lens (maintaining field of view for the physician) and operative field of any blood obstructing the anatomical landmarks, clear the operative field of blood, fungal or other obstructing debris. In further aspects, the hand piece is universal and can work in conjunction with any desired scope size. In some embodiments, warm irrigant can also allow for paradoxical vasoconstriction for further hemostasis. The hand piece may be primarily used with endoscopes for various sinus procedures but could be applied and optimized for use during other endoscopic procedures. In other embodiments, a foot pedal assembly may be included which may be mechanical and not electronic. Mechanical architecture allows for continuous suction and irrigation without interruption. There may be two pedals and each can act as spring-loaded pinch valves. Irrigation allows for cleaning the scope lens and irrigation of the operative field, suction also allows the clearing of the field. The suction and irrigation tubing runs from the wall, through the mechanical pedal pinch valves, and up the hand pieces where the lumens are joined into one.

By way of example, before an operation, the user (physician/nurse/tech) feeds the flexible suction and irrigation tubing that comes with the hand piece through the pinch valves. These pinch valves are normally closed until the user decides to open them by actuating the pedal. A sheath may be integrated into the hand piece and provides a barrier between the endoscope and tissue and also provides a lumen to guide fluid for irrigation and suction. The sheath's effective length may be adjustable to accommodate different endoscope models, a feature which is not known to be available in current solutions. Still further, hand piece accommodates multiple scopes from various manufactures with the adjustable sheath and Luer lock. In yet further aspects, flexible internal tubing and a separate internal plastic part with sealing O-ring provides the ability for the sheath to translate and fit different endoscopes. Furthermore, most current solutions are proprietary and scope specific.

In other aspects, the hand piece may be ergonomically designed to assist with relief of carpal tunnel pressure and fatigue. The hand piece geometry provides hand and carpal tunnel fatigue relief with an overmolded soft grip providing additional ergonomic comfort. It may be configured to allow supporting the scope/hand piece on the thenar eminence (proximal thumb web area) and also for comfort. The ergonomics can enable comfortable long-term use by the physician throughout multiple surgeries. The hand piece is configured in at least two forms: one with a larger, more comfort-oriented grip, and a second with a slimmer line hand piece, allowing for multiple user adaptations. In still further aspects, the geometry of each hand piece may be optimized for use during sinuplasty. In particular, the orientation of the grip and the orientation of the fluid port can be placed to optimize the physician's working area.

The description herein describes an endoscope system that can deliver a continuous flow of irrigation fluid to an operative field to clean the endoscope lens and provide the application of a fluid such as, for example, water or an antibiotic irrigation fluid. The system also includes an adjustable length sheath, for example, to accommodate different scope lengths as well as different diameters, a common port for suction and irrigation. The system includes hand grips and soft gripping surface that provide comfort for the user. The adjustable sheath may allow the device to engage with endoscopes having different lengths. In some embodiments, the device may use different interchangeable sheaths having different sizes and features.

In various embodiments, the disclosed apparatus, devices and systems comprise a sheath adjustment mechanism. In some embodiments, the sheath adjustment mechanism may be a coupler push button or the like. The system may include a valve assembly that is mechanical and provides an economic alternative to electromechanical valves that fail to provide a substantially continuous flow of irrigation and suctioned fluids. In this regard, the valve assembly in the illustrated exemplary embodiment includes foot pedals to operate the system.

In further aspects, the various embodiments of the disclosed apparatus, devices and systems include one or more of the following patentable features: adjustable scope sheath via a slider mechanism; Suction/Irrigation via Collar/Attachment; Hand piece ergonomic design/form covering; universal scope fitting; irrigation mechanism; irrigation motor/mechanism/foot pedal; and Scope Sheath—viable diameter; universal diameter and length adjustment.

In still further aspects, the various embodiments of the disclosed apparatus, devices and systems provide one or more the following advantages: Decrease in surgical time because of improved field of view; Improves time of surgery since it decreases time of cleaning scope; Irrigation of surgical field improves visibility and safety of procedure; Irrigation helps blood control; Increases suction of field; Irrigation of sinus cavity and antibiotic solution; Increases safety; Expands scope of possible dissection; Multiple scope sizes (universal); Suction via common Sheath containing the scope; and Sheath also has access to the surgical field.

In various aspects, the present disclosure provides various embodiments and configurations of an endoscopic apparatus. In an aspect, an endoscopic apparatus may comprise: a body portion having an inner cavity operative to engage and retain an endoscope; a first tubing portion having a first distal end and a second distal end arranged in the inner cavity; a fluid port operative to engage the first distal end of the first tubing portion; a flow assembly operative to engage the second distal end of the first tubing portion and a portion of the endoscope; and a hand grip portion arranged on the body portion.

In further aspects, the endoscopic apparatus may be embodied as a surgical hand piece apparatus for endoscopic procedures. In some embodiments, the apparatus may include one or more of: a body portion configured to receive and retain an endoscope; an adjustable sheath coupled to the body portion, wherein the adjustable sheath is configured to accommodate endoscopes of varying lengths; a combined suction and irrigation port integrated into the body portion; a mechanical foot pedal assembly communicatively connected to the combined suction and irrigation port, the mechanical foot pedal assembly including spring-loaded pinch valves for controlling fluid flow; and/or an ergonomic hand grip portion formed on the body portion, the ergonomic hand grip portion including a soft overmolded material designed to reduce carpal tunnel pressure and fatigue during use. The adjustable sheath may include a Luer lock mechanism for securing the endoscope. The combined suction and irrigation port may comprise a single Luer lock or barbed fitting. In further aspects, the mechanical foot pedal assembly may be non-electronic. Still further, the spring-loaded pinch valves may be color-coded for easy identification of suction and irrigation controls. The ergonomic hand grip portion may be designed to support the thenar eminence of a user's hand. The body portion further may comprise a rigid structure with a soft rubber overmold. The adjustable sheath may also be configured to provide a fluid flow path for both irrigation and suction fluids. Still further, the mechanical foot pedal assembly may include a heavy base for stability during operation. The adjustable sheath can also be configured to translate relative to the body portion to accommodate different endoscope models. The ergonomic hand grip portion may also include a thenar rest to allow support of the hand piece on the thumb web; and/or may be designed to provide carpal tunnel fatigue relief.

The mechanical foot pedal assembly may be configured to return to a default position that closes the spring-loaded pinch valves when not engaged; to provide continuous control over the flow of fluids; and/or to operate the spring-loaded pinch valves independently for suction and irrigation. The combined suction and irrigation port may be configured to allow continuous flow of fluids without interruption. Still further, the body portion may be configured to be universally compatible with multiple endoscope manufacturers. The ergonomic hand grip portion may be optimized for use during sinuplasty procedures. The body portion may include a flow valve assembly operative to direct fluid flow through the adjustable sheath. The body portion and the adjustable sheath may also be configured to allow for quick disassembly for cleaning and sterilization.

In other embodiments, the apparatus may include one or more of: a body configured to accommodate an endoscope; an ergonomic grip portion designed to conform to the contours of a user's hand; an integrated adjustable sheath for encasing a portion of the endoscope and allowing length adjustment; a combined suction and irrigation port connected to the body for providing fluid flow and suction through the endoscope; and/or a thenar rest positioned to support the hand piece on the user's thenar eminence during use. In further aspects, the ergonomic grip portion may include a soft grip material over a hard molded housing to enhance comfort and reduce hand fatigue, and/or may be designed to assist with the relief of carpal tunnel pressure during prolonged surgical procedures. The integrated adjustable sheath may include a Luer lock mechanism for securing the endoscope at various lengths. The combined suction and irrigation port may be designed to maintain a continuous flow of irrigation fluid and suction without interruption. The thenar rest may be contoured to provide stability and control of the hand piece during endoscopic maneuvers.

In further aspects, the apparatus may further comprise a fluid channel within the adjustable sheath that is optimized to prevent clogging and ensure clear visibility through the endoscope. The body of the hand piece may be constructed from materials compatible with sterilization processes used in medical facilities. The ergonomic grip portion may include a textured surface to improve grip and prevent slippage, and/or the body of the hand piece may be designed to be lightweight to minimize user fatigue during extended use. Still further, the ergonomic grip portion may be available in multiple sizes to accommodate different hand sizes of users. The thenar rest may include a cushioned pad for added comfort during use. In still further aspect, the body of the hand piece may include visual indicators for alignment with the endoscope. The integrated adjustable sheath may be detachable for easy cleaning and replacement. The ergonomic grip portion is designed to allow for multiple user adaptations, including grip size and shape. The body of the hand piece may include an integrated channel for passing additional surgical instruments alongside the endoscope. The combined suction and irrigation port may include a single Luer lock or barbed fitting for streamlined connection to fluid sources. The ergonomic grip portion may be designed to distribute pressure evenly across the user's hand to prevent pressure points, and the body of the hand piece may be designed to facilitate a neutral wrist position to reduce the risk of repetitive strain injuries. The body of the hand piece may also include a modular design for customization and adaptability to various endoscopic procedures.

In further aspects, the apparatus may include fluid control features. For example, in another aspect, the apparatus may include one or more of: an integrated adjustable sheath designed to accommodate an endoscope and provide a fluid pathway for irrigation and suction; a combined suction and irrigation port operatively connected to the adjustable sheath; a valve mechanism within the adjustable sheath for regulating the flow of irrigation fluid and suction; and/or a control interface accessible on the surgical hand piece for user manipulation of the valve mechanism. In further aspects, the valve mechanism may include a spring-loaded pinch valve for each of the irrigation and suction pathways. The combined suction and irrigation port can include a single connection point for both irrigation fluid supply and suction evacuation. The control interface may comprise a mechanical actuator linked to the valve mechanism for precise flow control, and/or include ergonomic features to facilitate operation with minimal hand movement, and/or designed to prevent backflow and cross-contamination between the pathways. The adjustable sheath may include a transparent section for visual monitoring of fluid flow. The valve mechanism may be configured to maintain a continuous and uninterrupted flow when in an open position, and/or may be replaceable for maintenance and longevity of the apparatus. Still further, the adjustable sheath and the valve mechanism may be constructed from materials resistant to common sterilization processes.

In yet further aspects, the apparatus may include visualization features. For example, in another aspect, the apparatus may include one or more of: an endoscope with an integrated camera for capturing images within a patient's body; an adjustable sheath configured to encase a portion of the endoscope and provide a clear fluid pathway for maintaining lens cleanliness; a fluid control system integrated into the adjustable sheath for delivering an irrigation fluid to the distal end of the endoscope to enhance image clarity; and/or a connection interface for transmitting captured images from the integrated camera to an external display. The adjustable sheath may include a transparent section for direct visual inspection of the endoscope lens, and/or may be designed to minimize glare and reflections that may interfere with imaging quality. The fluid control system may comprise a valve mechanism for regulating the flow of the irrigation fluid to prevent lens obstruction, and/or may be designed to deliver a steady stream of irrigation fluid without disrupting the captured images. The connection interface may include a high-definition video output compatible with various external display technologies, and/or support real-time image processing and enhancement features. The integrated camera may be capable of capturing images in low-light conditions within the patient's body, and/or include a zoom function for detailed visualization of the operative field. Still further the adjustable sheath and fluid control system may be autoclavable for sterilization without damaging the integrated camera.

In even further aspects, the apparatus may include image capture and transmission features. For example, in another aspect, the apparatus may include one or more of: an endoscope equipped with an electronic image sensor for capturing visual data; a data processing unit integrated within the endoscope for converting visual data into electronic image signals; a transmission module for sending electronic image signals to an external processing system; and/or a power supply mechanism for providing electrical energy to the electronic image sensor and data processing unit. The data processing unit can include an image signal processor for enhancing image quality, and/or the transmission module can include a wireless communication interface for remote image signal transmission. The power supply mechanism may comprise a rechargeable battery integrated into the endoscope handle. Still further, the electronic image sensor may be capable of capturing high-resolution images suitable for diagnostic purposes, and the transmission module may be compatible with standard medical image storage and communication protocols. The data processing unit may include real-time image stabilization features to compensate for motion during the endoscopic procedure, and the power supply mechanism may include a low-battery indicator to alert the user of the need for recharging or replacement. The transmission module may also supports high-bandwidth data transfer to minimize latency in image display, and the electronic image sensor may include a light-sensitive chip for capturing images in variable lighting conditions within the patient's body.

In even further aspects, the apparatus may include wireless communication and connectivity features. For example, in another aspect, the apparatus may include one or more of: an endoscope with an integrated wireless transmitter for sending visual data; a remote visualization unit capable of receiving and displaying the visual data transmitted by the endoscope; a control interface on the endoscope for wireless operation of endoscopic functions; and/or a synchronization module for establishing and maintaining a secure wireless connection between the endoscope and the remote visualization unit. The integrated wireless transmitter may operate on a frequency band reserved for medical devices to minimize interference, and the remote visualization unit may include a high-definition display for detailed image analysis. The control interface may allow for remote adjustment of endoscopic settings such as light intensity and image capture modes, and/or the synchronization module may utilize encryption protocols to ensure the privacy and security of transmitted data. The apparatus may further comprise a wireless receiver integrated into the endoscope for receiving control signals from the remote visualization unit, and the remote visualization unit may be equipped with touch-screen capabilities for intuitive control and navigation. The endoscope may also include a rechargeable battery with wireless charging capabilities for continuous operation. Still further, the synchronization module may support multiple wireless communication standards for compatibility with various external devices, and/or the control interface may include haptic feedback to confirm successful operation of remote commands.

In still further aspects, the apparatus may be modular. For example, in some embodiments, the apparatus may include one or more of: a main body with a universal interface for attaching various ergonomic grip modules; a selection of interchangeable ergonomic grip modules designed to accommodate different hand sizes and preferences; a detachable adjustable sheath component for encasing an endoscope, with variable length settings; a set of interchangeable fluid control modules for customizing irrigation and suction capabilities; and a quick-connect mechanism for attaching and detaching the ergonomic grip modules and fluid control modules to the main body. Each ergonomic grip module may include a distinct texture or material to provide various tactile experiences and may further comprise a storage case for organizing and maintaining the selection of interchangeable modules. The detachable adjustable sheath component may include a range of diameters to fit different endoscope models. The fluid control modules can include options for manual or automated flow regulation, and/or are color-coded to facilitate identification and selection based on procedural requirements. The quick-connect mechanism may comprise a locking collar that ensures secure attachment while allowing for easy module exchange. The main body may include an integrated electronic interface for connecting to endoscope imaging systems, and/or may be constructed from a material compatible with high-level disinfection and sterilization processes. The ergonomic grip modules may be designed to reduce the risk of repetitive strain injuries through ergonomic assessment. In other embodiments, the apparatus may include one or more of: a base surgical hand piece with a universal connector for attaching various endoscopic tools; a set of interchangeable endoscope modules, each designed for specific surgical procedures; a customizable fluid control system with modular components for irrigation and suction tailored to procedure-specific requirements; a selection of ergonomic grips of varying sizes and shapes to accommodate user preference and procedure demands; and/or a user interface module that can be configured with procedure-specific control settings. In still further aspects, the apparatus may further include one or more of: the universal connector includes a quick-release mechanism for rapid tool exchange; each interchangeable endoscope module is optimized for a particular field of view and depth of focus; customizable fluid control system includes interchangeable valves and pumps for different flow rate requirements; ergonomic grips featuring a tool-free attachment system for easy customization; a user interface module that includes a touch screen that can be programmed with custom control profiles; a selection of sheaths with different diameters and lengths for various endoscope modules; base surgical hand piece that include an integrated power supply module that can be swapped for different battery capacities; fluid control system modules that are color-coded for easy identification and matching with corresponding surgical procedures; user interface module that is capable of wirelessly syncing with a central database to update control settings and software; universal interface that includes multiple video output formats to ensure compatibility with high-definition and 3D surgical monitors; adaptable power supply that features an automatic voltage adjustment system for international use; surgical environment sensors that detect lighting conditions, temperature, and humidity to optimize the apparatus performance; communication module that supports wireless and wired connectivity protocols, including Bluetooth, Wi-Fi, and Ethernet; sterilizable docking station that includes RFID or barcode scanning for tracking and inventory management; a modular accessory kit with various endoscopic tools for different surgical specialties; communication module that is capable of receiving software updates and configuration changes remotely; universal interface designed to connect with robotic surgical systems for enhanced control during minimally invasive procedures; and/or docking station that includes a user authentication system to ensure secure access to the apparatus.

In further aspects, the apparatus may be sterilizable. For example, the apparatus may comprise one or more of: a surgical hand piece constructed from materials resistant to high-temperature sterilization processes; an endoscope with components and assemblies designed to withstand chemical disinfection; a detachable sheath made from a biocompatible material suitable for autoclave sterilization; and/or a fluid control system with components that maintain functionality after exposure to sterilizing agents. In various further aspects, the apparatus may include one or more of: a surgical hand piece materials include medical-grade stainless steel and high-temperature polymers; the endoscope components are sealed to prevent damage from liquid disinfectants; wherein the detachable sheath is coated with an antimicrobial layer to enhance sterilization efficacy; the fluid control system includes valves and seals made from silicone or other elastomers compatible with sterilization processes; a wireless communication module with encapsulation to protect electronic components during sterilization; the endoscope includes a removable camera module designed for separate sterilization; the materials used for the surgical hand piece and detachable sheath are certified for repeated sterilization cycles without degradation; the fluid control system is modular, allowing for disassembly and individual sterilization of components; the apparatus includes indicators that change color or appearance to confirm successful sterilization.

In further aspects, the apparatus may include integrated fluid control. For example, the apparatus may include one or more of: a surgical hand piece with an embedded fluid control system for managing irrigation and suction during endoscopic procedures; a dual-channel tubing system within the hand piece, one for delivering irrigation fluid to the operative field and another for suction removal of fluids; a valve assembly operable to independently control the flow rate of irrigation and suction; a fluid reservoir connectable to the irrigation channel for supplying the irrigation fluid; and/or a waste collection container connectable to the suction channel for collecting fluids removed from the operative field. In still further aspects, the apparatus may further include one or more of: the valve assembly includes adjustable controls for fine-tuning the flow rates of irrigation and suction in real-time; the fluid reservoir is designed to maintain a sterile environment for the irrigation fluid; the waste collection container includes a filter mechanism to separate debris from the suctioned fluids; a pressure regulation system to ensure consistent irrigation flow and suction vacuum levels; a dual-channel tubing system made from a flexible, biocompatible material that resists kinking and blockage; the surgical hand piece includes a user interface with indicators for monitoring fluid levels and flow rates; a fluid control system capable of simultaneous irrigation and suction without cross-contamination; a valve assembly operable via a foot pedal mechanism, allowing hands-free control by the user; and/or the fluid reservoir and waste collection container easily detachable for quick replacement during procedures.

In further aspects, the apparatus may be ergonomically designed. For example, in further embodiments, the apparatus may comprise one or more of: a surgical hand piece with a contoured grip conforming to human hand ergonomics to reduce fatigue during prolonged use; a weight distribution system within the hand piece to balance the apparatus and minimize strain on the user's wrist and arm; a textured surface on the grip areas to enhance grip stability and prevent slippage; adjustable support features to accommodate various hand sizes and grip styles; and/or a user interface positioned to allow intuitive access to controls without requiring significant hand movement. In other aspects, the apparatus may further include one or more of: a contoured grip includes a padded overlay for additional comfort; a weight distribution system adjustable to adapt to different endoscope weights and lengths; textured surface comprising a non-slip silicone or rubber material; adjustable support features that may include a thumb rest and finger grooves; a user interface that may include touch-sensitive controls that respond to light pressure; a detachable wrist support to maintain the hand in a neutral position; the hand piece designed to reduce pressure points and prevent carpal tunnel syndrome; grip areas that may be are thermally insulated to maintain a comfortable temperature during use; and/or a hand piece that may include visual indicators for ergonomic adjustments to guide the user to an optimal grip configuration.

In further aspects, the disclosure provides an adjustable sheath apparatus and an apparatus that may include an adjustable sheath, which can be, for example, be used with a surgical hand piece in endoscopic procedures. The adjustable sheath may comprise one or more of: a sheath body configured to encase a portion of an endoscope; an adjustment mechanism integrated into the sheath body for altering the effective length of the sheath to accommodate endoscopes of varying lengths; a fluid channel within the sheath body designed to direct irrigation fluid towards the distal end of the endoscope and to facilitate suction from the proximal end; a securement feature for maintaining a fluid-tight connection between the sheath body and the endoscope; and/or an attachment interface for removably coupling the adjustable sheath apparatus to the surgical hand piece. In still further aspects, the adjustable sheath may include one or more of: an adjustment mechanism that includes a sliding collar that locks into place at various positions along the sheath body; securement feature that comprises a Luer lock mechanism compatible with standard endoscope designs; fluid channel configured to maintain separation between the irrigation fluid and the suctioned materials; attachment interface that includes a quick-release mechanism for rapid detachment from the surgical hand piece; sheath body constructed from a biocompatible and corrosion-resistant material suitable for medical use; sheath body that includes a transparent section for visual monitoring of the fluid channel; adjustment mechanism operable with a single hand to facilitate ease of use during surgical procedures; securement feature that includes an internal sealing element, such as an o-ring, to enhance the fluid-tight connection; sheath body designed to provide structural support to the endoscope while allowing flexibility for maneuvering; attachment interface compatible with multiple models of surgical hand pieces; sheath body that includes graduated markings to indicate the adjusted length of the sheath; sheath body autoclavable for sterilization between uses; fluid channel that includes a valve mechanism to control the flow rate of irrigation fluid; sheath body designed to minimize optical distortion when used in conjunction with the endoscope; sheath body that includes an ergonomic profile to facilitate handling and positioning during the endoscopic procedure; sheath body designed to reduce glare and reflections that may interfere with the endoscopic imaging; sheath body that includes a textured exterior surface to improve grip and control during manipulation; sheath body designed to be disposable for single-use applications; sheath body that includes an integrated channel for passing additional instruments alongside the endoscope

In further aspect, the disclosure provides mechanical foot pedal assembly and an apparatus that may include a mechanical foot pedal assembly, which can be, for example, be used with a surgical hand piece apparatus during endoscopic procedures. The mechanical foot pedal assembly for controlling suction and irrigation may comprise one or more of: a base structure configured to support a user's foot actions; a first pedal pivotally attached to the base structure and associated with a first spring-loaded pinch valve for controlling irrigation fluid flow; a second pedal pivotally attached to the base structure and associated with a second spring-loaded pinch valve for controlling suction flow; a set of color-coded indicators on the first and second pedals for distinguishing between the irrigation and suction controls; and/or a tubing retention system integrated into the base structure for securing the flexible suction and irrigation tubing from the surgical hand piece apparatus. In still further aspects, the mechanical foot pedal assembly may comprise one or more of: base structure that includes a non-skid surface to prevent movement during operation; first and second spring-loaded pinch valves configured to provide continuous and uninterrupted fluid flow when actuated; first and second pedals configured to return to a default position that closes the pinch valves when not engaged by the user; color-coded indicators that comprise visually distinct colors that correspond to universal color standards for medical irrigation and suction; tubing retention system that includes adjustable clamps to accommodate various tubing diameters; a heavy-duty construction of the base structure to provide stability and durability during repeated use; spring-loaded pinch valves that are replaceable for maintenance and longevity of the assembly; first and second pedals ergonomically designed to minimize foot strain during extended surgical procedures; base structure that includes a carrying handle for portability; base structure constructed from a material resistant to common sterilization processes; first and second pedals that include textured surfaces to enhance grip and control; base structure designed to facilitate easy cleaning and disinfection; tubing retention system designed to prevent accidental dislodgement of the tubing during use; assembly compatible with a range of surgical hand piece apparatuses from various manufacturers; assembly that includes a locking mechanism to maintain the selected flow rate of irrigation fluid or suction; assembly designed to operate without the need for electrical power; assembly that includes a feedback mechanism to provide tactile or auditory confirmation of valve actuation; assembly designed to be operated by either foot of the user; and/or assembly that includes a modular design for easy replacement of individual components.

These apparatus claims are focused on the mechanical foot pedal assembly, detailing its structure, functionality, and features that contribute to controlling suction and irrigation during endoscopic procedures. Each dependent claim adds specific features or enhancements that refine the assembly, potentially increasing the scope of patent protection for this aspect of the invention.

### Device and System Configuration

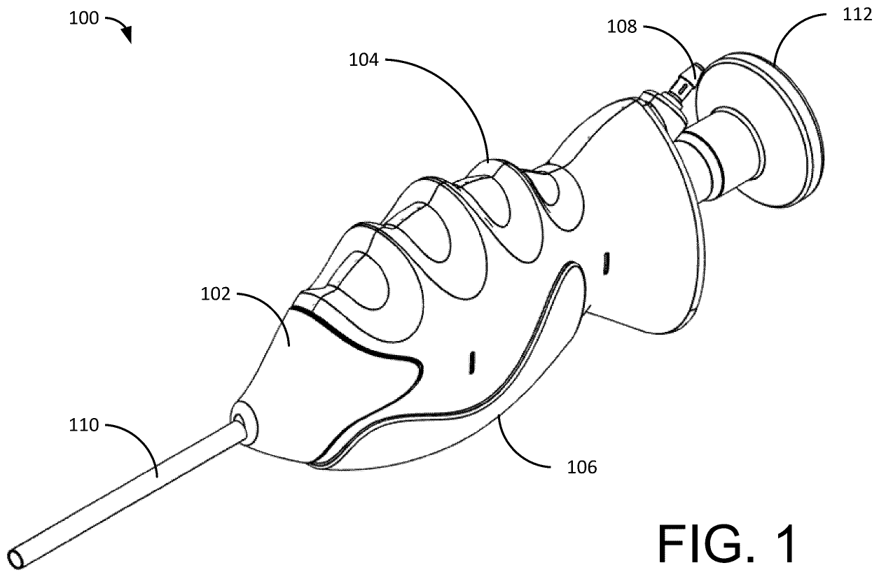

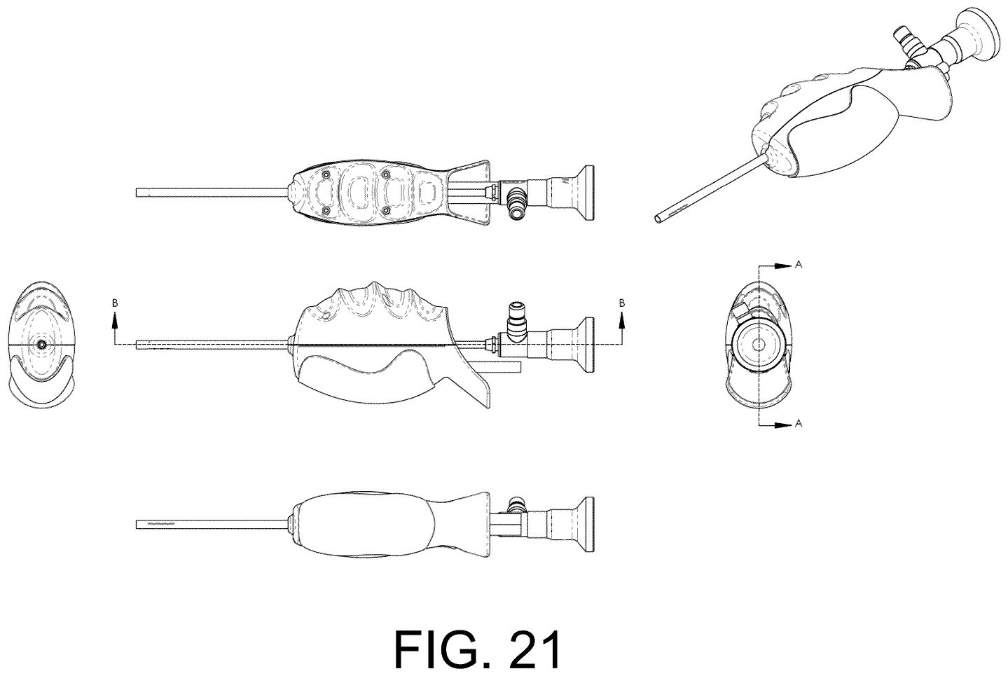

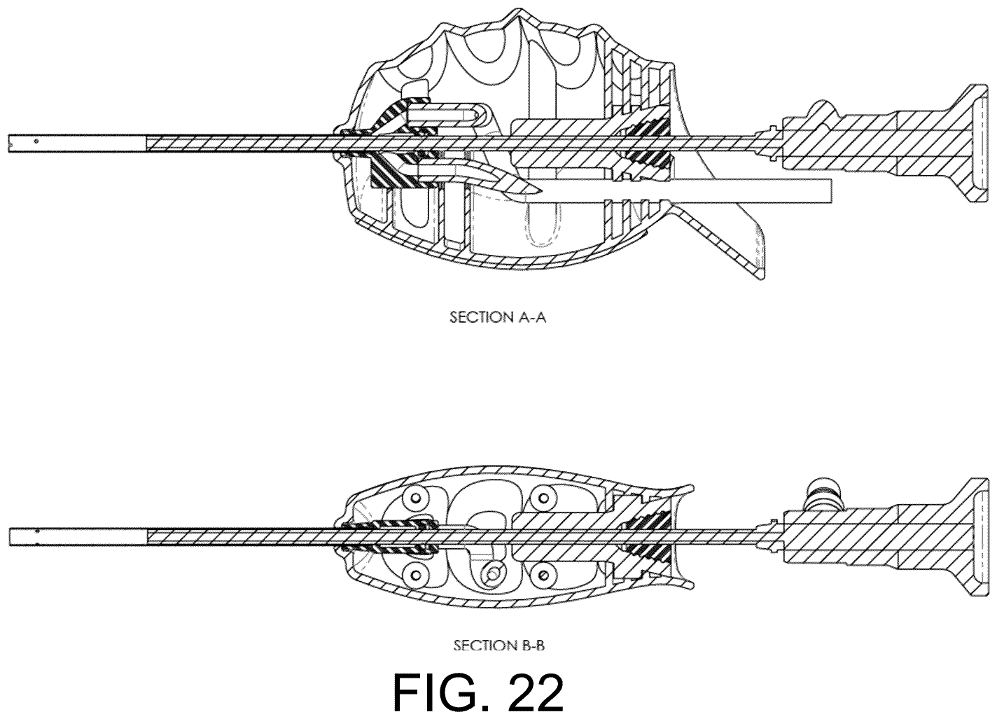

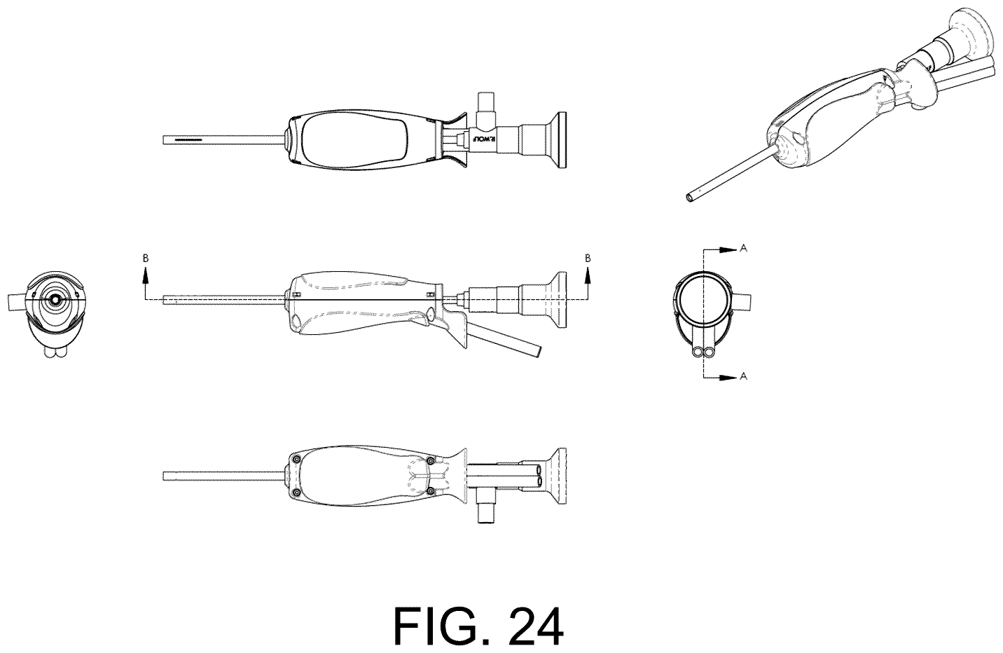

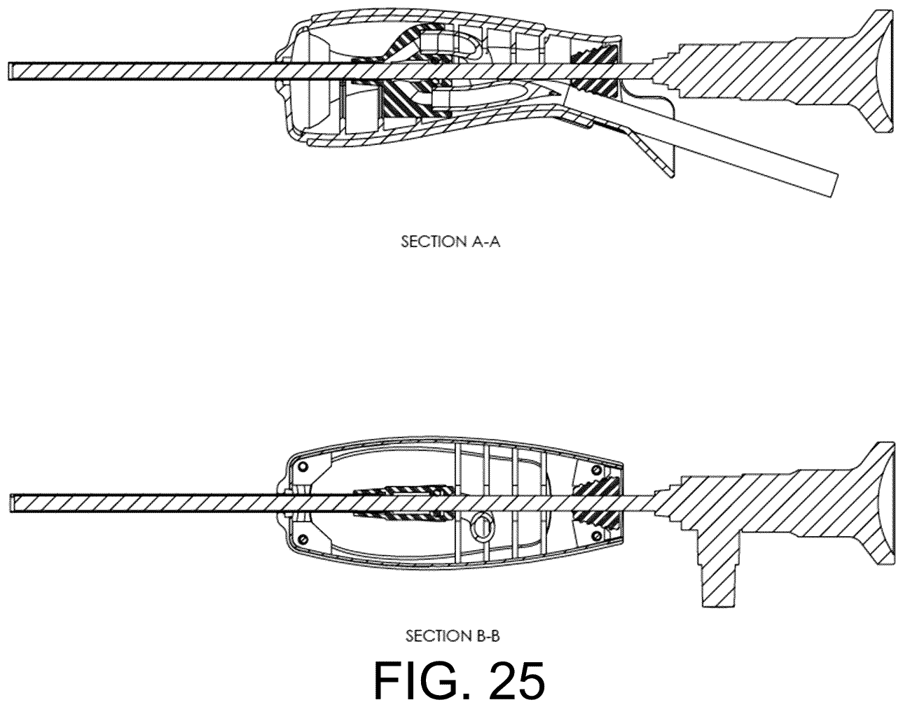

According to various aspects of the invention, the disclosed apparatus, devices and systems of the present disclosure can comprise multiple configurations. FIGS. 1-26 illustrate non-limiting examples of embodiments of operating environments, mechanisms, and components for the disclosed assemblies, devices, and systems. Although the operating environments, mechanisms, and components are disclosed with specific functionality, it should be understood that functionality may be shared between mechanisms and/or components, with some functions split between mechanisms and/or components, while other functions duplicated by the mechanisms and/or components. Furthermore, the name of the mechanisms and/or components should not be construed as limiting upon the functionality of the mechanisms and/or components. Moreover, each stage in the claim language can be considered independently without the context of the other stages. Each stage may contain language defined in other portions of this specifications. Each stage disclosed for one mechanism and/or component may be mixed with the operational stages of another mechanism and/or component. Each stage can be claimed on its own and/or interchangeably with other stages of other mechanisms and/or components.

By way of non-limiting example, FIG. 1 illustrates a planar view of an apparatus 100 for endoscopic procedures in accordance with an example embodiment of the present invention. The apparatus 100 in the illustrated embodiment is arranged to provide both suction and irrigation in the operative field. The apparatus 100 includes a body portion 102 that may be formed from any suitable material such as, for example, a metal, plastic, or composite type material. The first grip area 104 and a second grip area 106 are arranged on the body portion 102. The apparatus 100 includes a tubing retaining portion 108. An endoscope 112 is shown engaging the body portion 102. A sheath 110 is arranged about a portion of the endoscope 112. The sheath 110 is substantially tubular, and may be formed from a material such as, for example, a metal, plastic, or polymer material.

In some embodiments, the irrigant or irrigation fluid, such as a saline solution, may include an antibiotic or antimicrobial solution to improve the recovery of a patient. In this regard, the valve system of the endoscopic apparatus 100 (discussed below) includes pinch nasal valves to provide a continuous suction or irrigation so that the operative field may be irrigated with a warm irrigant to provide for paradoxical vasoconstriction for further hemostasis, clearing of debris, blood, puss, and the like.

In further aspects, the tube retaining portion of the illustrated embodiment may include a lure lock, however, other embodiments may include any type of suitable arrangement to secure tubing to the apparatus 100 such as, for example, a barbed connector, or the like.

In various further aspects, the disclosed apparatus, devices and systems may be configured to be universal and may be used with any scope diameter size, for example, the standard diameter of the scope of about 3 to 4 mm. In yet further aspects, the sheath may comprise any medical grade material, for example, a rigid polymer or stainless steel, or the like.

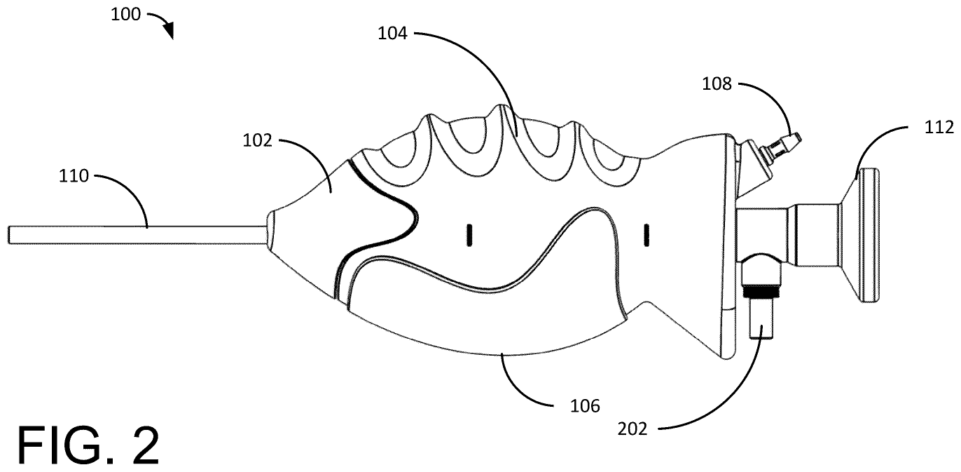

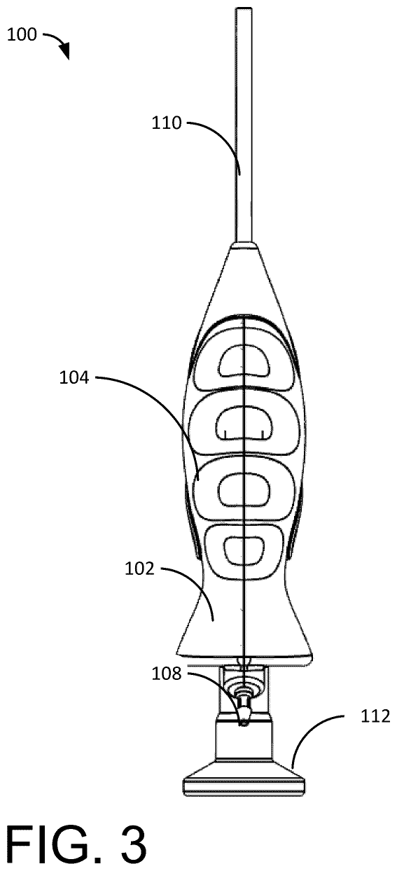

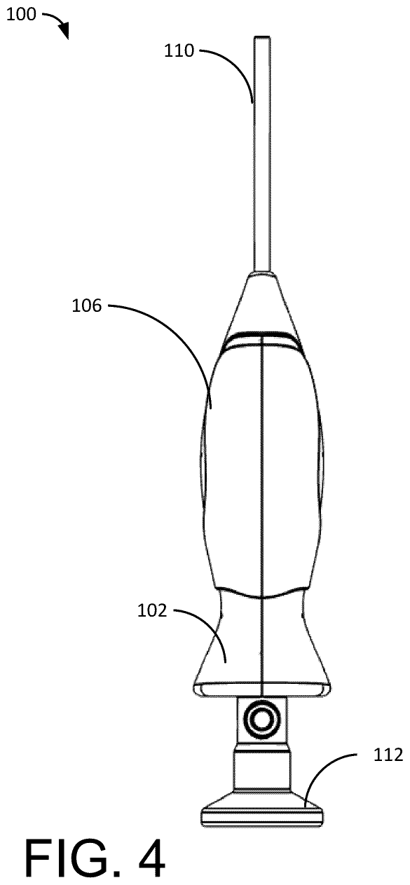

In an aspect, FIG. 2 illustrates a side view of the apparatus 100. In this regard, the endoscope 110 includes a coupling 202 that is operative to engage a light source (not shown). The endoscope 110 includes a view portion 204 that is operative to engage a camera (not shown). In further aspects, FIG. 3 illustrates atop view of the endoscopic apparatus 100. The first grip area 104 is shown having recesses sized and shaped to engage the fingers of a user. In this regard, the recesses in the first grip 104 area are arranged to facilitate a secure and comfortable grip for the user. The first grip area may include a plastic or deformable area to further provide padding or a cushion for the hand of a user. In still further aspects, FIG. 4 illustrates a bottom view of the apparatus 100. The illustrated exemplary embodiment includes the second grip area 106 that is operative to engage the region between the thumb and finger of a user. The second grip area 106, and in some exemplary embodiments, the first grip area 104, as discussed above may include a deformable or padded material that is operative to provide a comfortable grip for the user. In the illustrated exemplary embodiment, the bottom grip area 106 may fit between a thumb and forefinger of a user for a secure and comfortable grip.

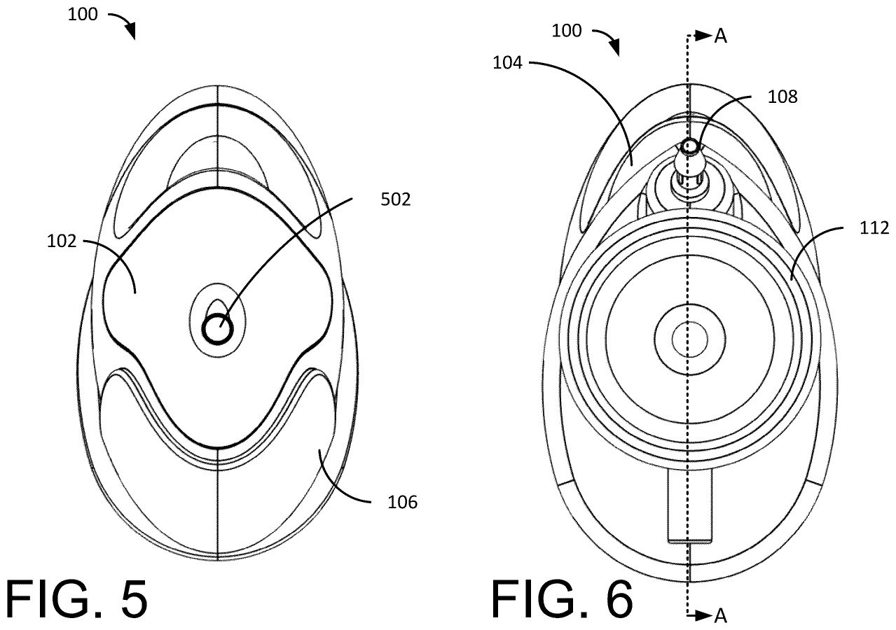

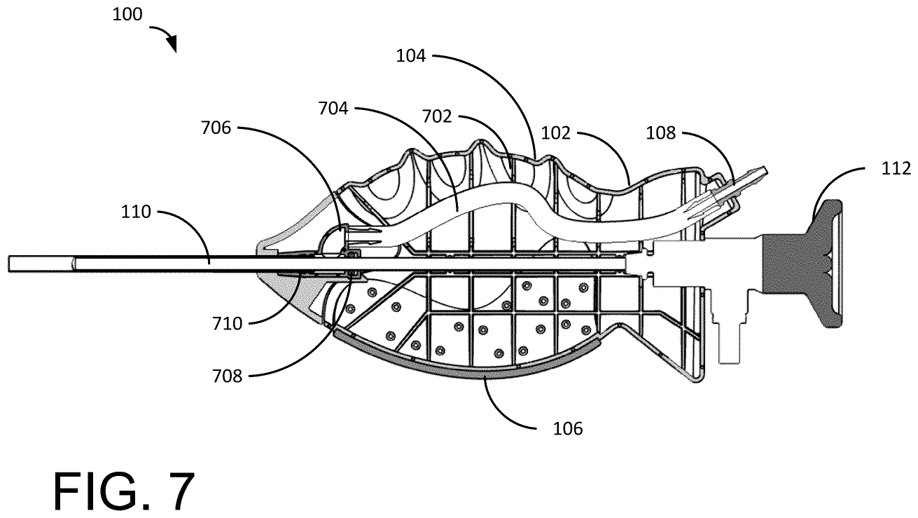

In further aspects, FIG. 5 illustrates a front view of the apparatus, and FIG. 6 illustrates a bottom view of the apparatus 100. The exemplary embodiment includes a lens 502 that is arranged on a first distal end of the scope 112. FIG. 5 further illustrates the second grip area 106 that is operative to engage the region between the thumb and fore finger of a user. In yet further aspects, FIG. 7 illustrates a cut-away view along the line A-A (of FIG. 6) of the apparatus 100. In this regard, the apparatus 100 includes a cavity 702 that is operative to receive the scope 112. The tube retaining portion 108 is shown extending through the body 102 and connecting with a substantially flexible tube portion 704 that is arranged in the cavity 702. One distal end of the tube portion 704 is connected to the tube retaining portion 108 such that fluid received by the tube retaining portion 108 passes through the tube retaining portion 108 and enters the tube portion 704. Another distal end of the tube portion 704 is connected to a coupler portion 706. The coupler portion 706 in the illustrated exemplary embodiment is operative to retain the distal end of the tube portion 704. The coupler portion 706 also includes a seal portion 708 that forms a seal between the coupling portion 706 and the sheath 110, the seal portion may include any suitable seal arrangement such as, for example, an O-ring or gasket arrangement.

In various aspects, apparatus 100 can define a fluid flow path that flows from the control valves (described below) through a length of tubing to the tubing retaining portion 108. The fluid passes through the tubing retaining portion 108 and into the tube portion 704 to the coupler portion 706. The coupler portion has an inner cavity 710 with the seal portion 708 impeding the flow of fluid into the cavity 702. The coupler portion includes an outlet orifice that is operative to direct a flow of fluid for an irrigation procedure, and the suction of fluid to clear from the operative field.

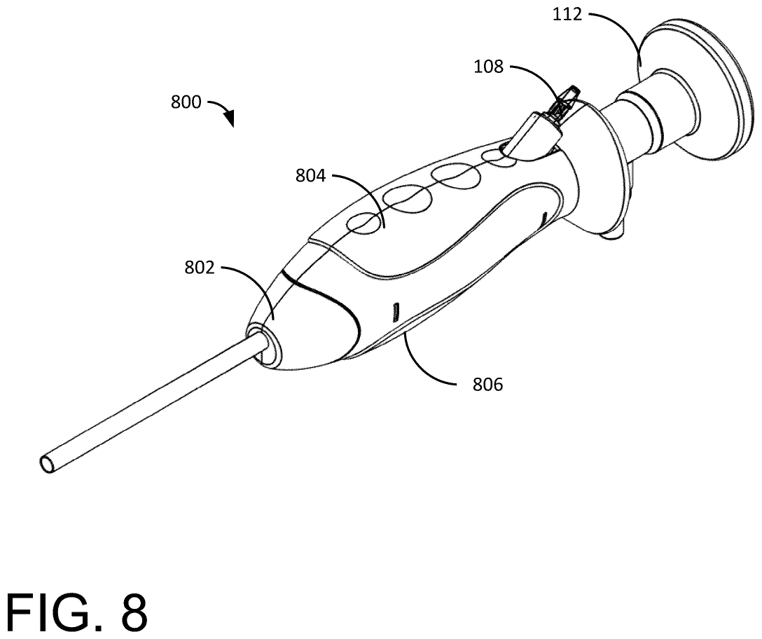

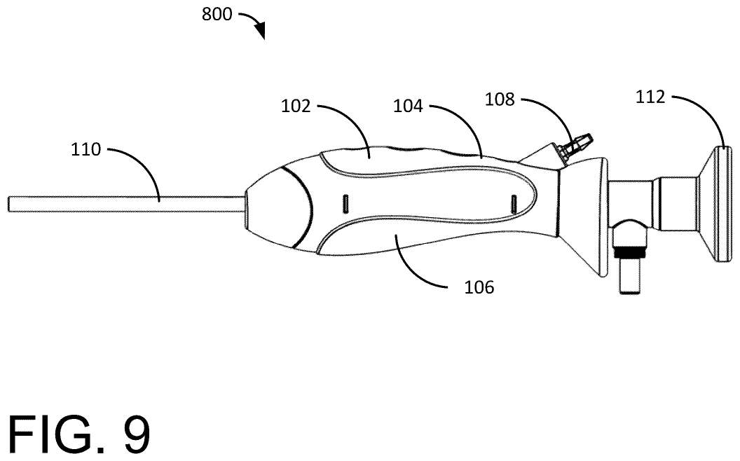

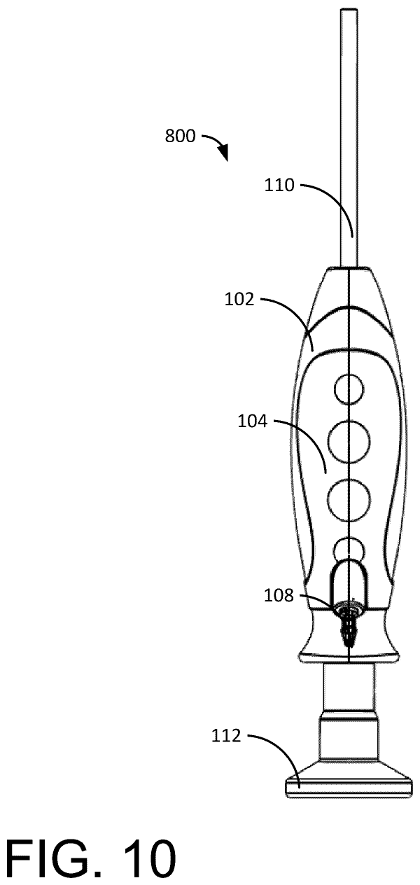

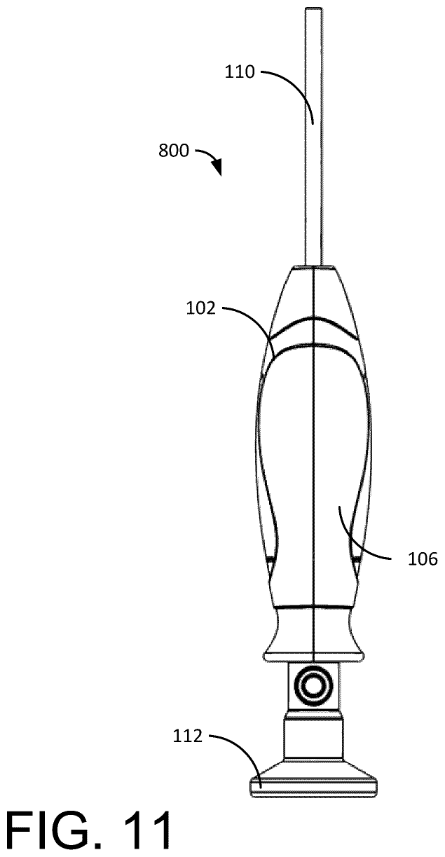

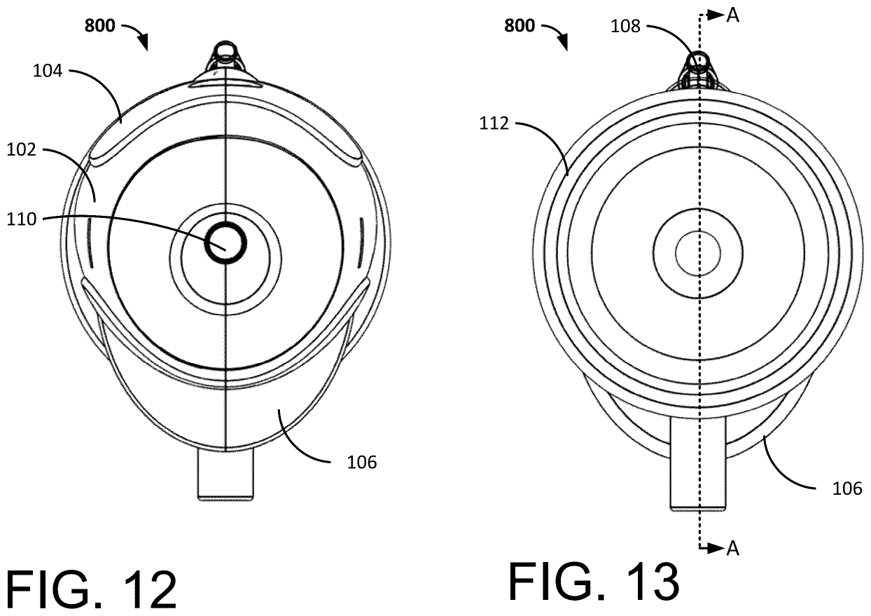

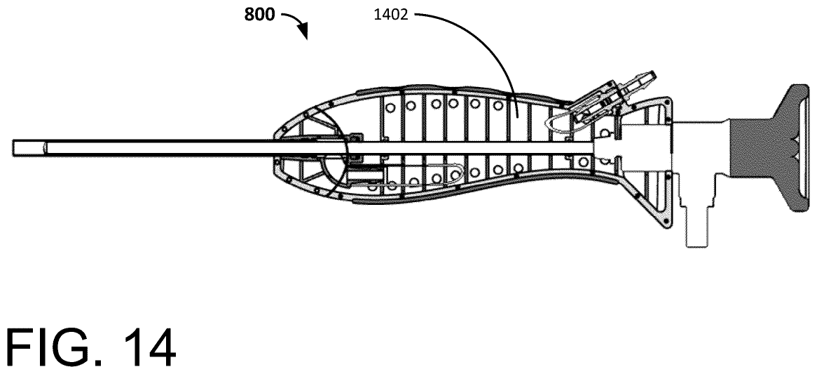

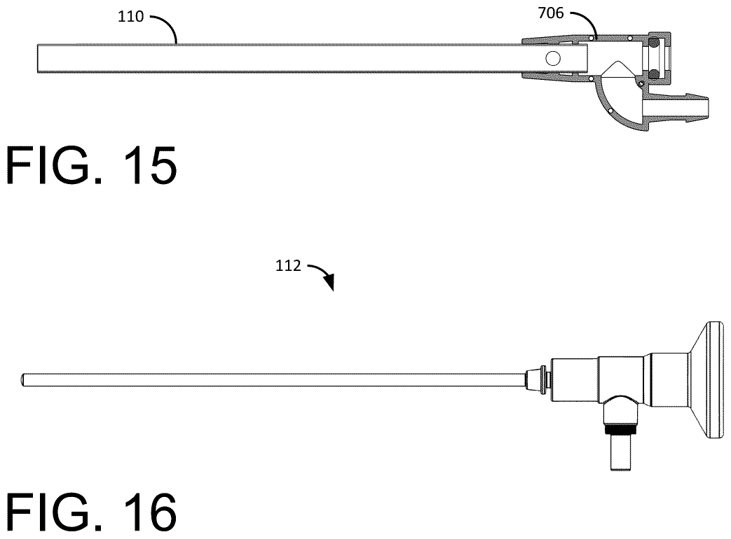

In another aspect, FIG. 8 illustrates a perspective view of an alternate exemplary embodiment of a endoscope apparatus 800. The apparatus 800 is similar to the endoscopic apparatus 100 described above however, the body portion 802 is a smaller size than the endoscopic apparatus 100 to accommodate some medical procedures where a smaller device is desired. The apparatus 800 includes a first handgrip portion 804 and a second handgrip portion 806 that provide cushioning and a comfortable grip for the user in a similar manner as discussed above. In further aspects, FIG. 9 Illustrates a side view of the apparatus 800, FIG. 10 illustrates atop view of the apparatus 800, FIG. 11 illustrates a bottom view of the apparatus 800, FIG. 12 illustrates a front view of the apparatus 800, FIG. 13 illustrates a rear view of the apparatus 800, and FIG. 14 illustrates a cut-away view along the line A-A (of FIG. 13). The apparatus 800 includes a cavity 1402 that is similar to the endoscopic apparatus 100 above. However, the cavity in FIG. 14 has a smaller volume as compared to the endoscopic apparatus 100. FIG. 15 illustrates a cut-away view of the sheath 110 and the coupler portion 706. FIG. 16 illustrates a side view of an example of an endoscope 112 for use in accordance with the present invention.

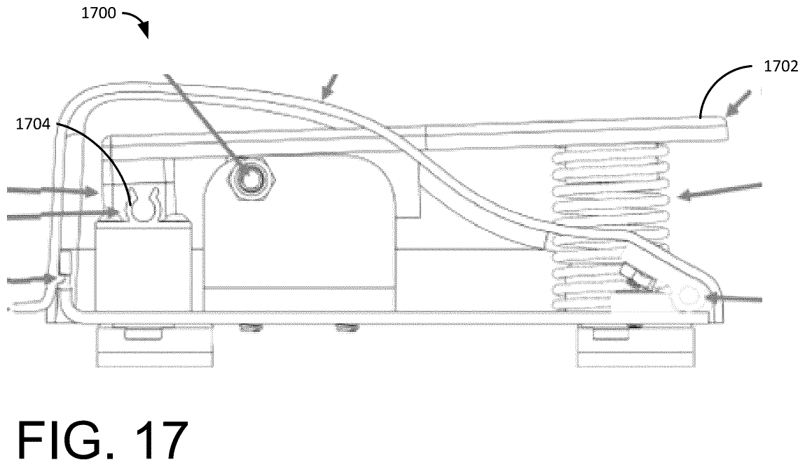

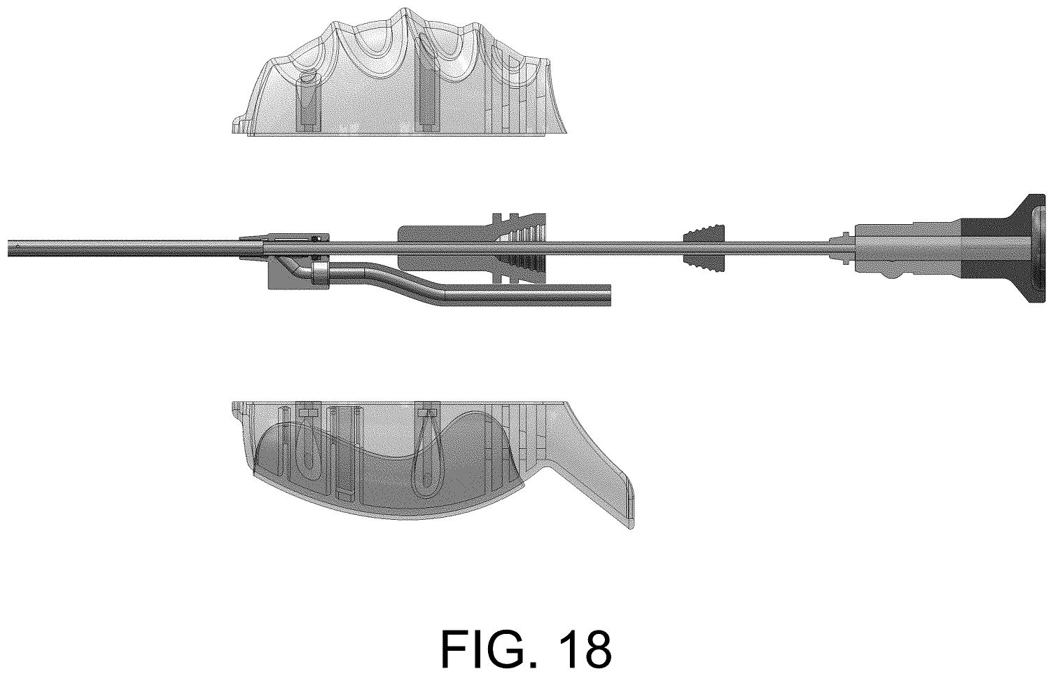

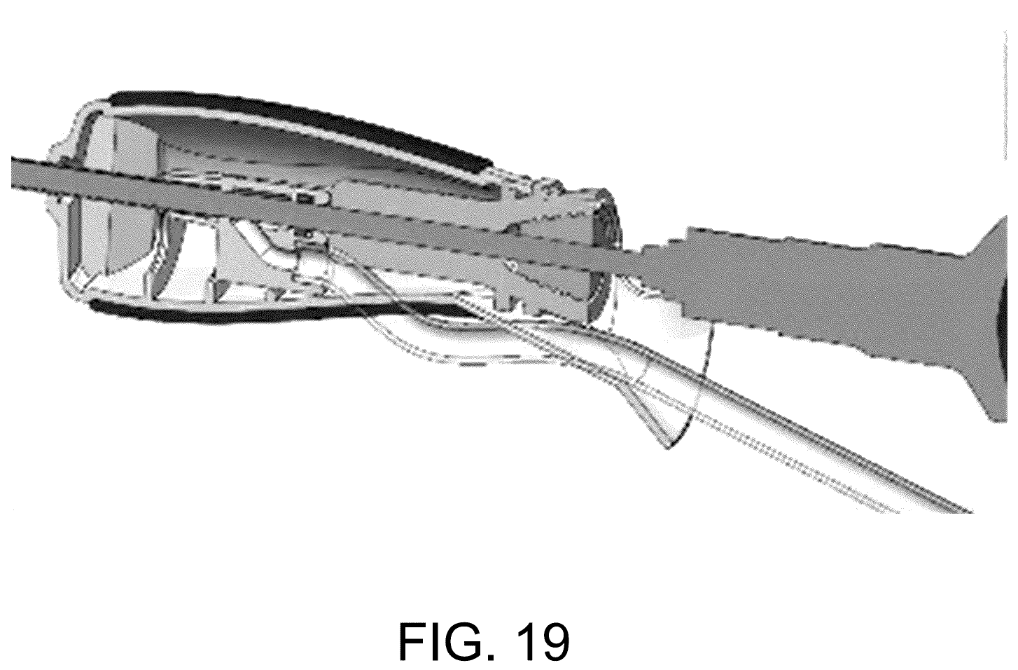

In further aspects, FIG. 17 illustrates partially cut away view of an exemplary embodiment of a control assembly 1700. The control assembly 1700 of the illustrated exemplary embodiment includes a pair of levers 1702 that are arranged to be operated by the feet of a user. A lever 1702 is operative to control the system to provide suction while a corresponding lever 1702 (not shown) is operative to control irrigation. The assembly 1700 includes valves that may have any suitable design such as, for example, pinch valves or another type of mechanical or electrical valve. In the exemplary embodiment, the levers 1702 are operative to move about an axis of rotation to open pinch valves 1704 that are arranged with corresponding tube that is communicatively connected to the apparatus 100 (of FIG. 1). The arrangement of the control assembly 1700 offers a device that allows a constant flow of fluid or a vacuum without using electronic components.

In various further aspects, FIGS. 18 and 19 illustrate yet another example embodiment of a device and apparatus in accordance with the present invention. The device in the illustrated embodiment is arranged to provide both suction and irrigation in the operative field.

In various aspects, the disclosed embodiments may comprise an endoscope and can be used for sinuplasty or similar procedures. The endoscope may extend through the entire body and comprise a camera and a light source at the end. In some embodiments, the system may comprise an ergonomic grip make it less fatiguing for the position during use in a procedure or surgery. In still further aspects, the ergonomic grip is configured to mate the fluid source, for example, the suctioning and irrigation features. By way of example, the system may be able to send fluid to the tip, for example to clean, then suction may be applied to pull the fluid back out. In some aspects, there may be a plurality of concentric tubes for sending fluid and suctioning used fluid. In still further aspects, the system allows suction and irrigation of field across the scope lens for cleaning as well as irrigation of surgical field.

In further aspects, a system according to the present disclosure comprises a multi-part stabilizing system (e.g., yellow and pink components in FIG. 18) comprising a female base portion and male insert configured to cooperate to assist in keeping the endoscope stationary relative to the handpiece. In some aspects, the male part may be a lower durometer material (e.g., a lower durometer rubber) and comprise a shape effective to insert into the female part of the stabilizing system. In other aspects, the lower durometer material enables increased friction against the surface it touches, such as the scope. In still other aspects, the male part may be rotated body configured to push into a mating part as shown in FIG. 19. In further aspects, the stabilizing system may comprise graduated amounts or stages that the insert can be inserted into the mating part. To this end, once the insert is all the way in the mating part, it can squeeze down and apply more friction to the to the scope and hold it in place. In still further aspects, the stabilizing system may comprise a threshold force that can overcome (e.g., by pulling) to unlock and allow a user to reset the scope angle and/or inserted scope depth. In still further aspects, the stabilizing system is configured to allow user to make and maintain real-time adjustment of an inserted scope, for example, scope angle and scope depth.

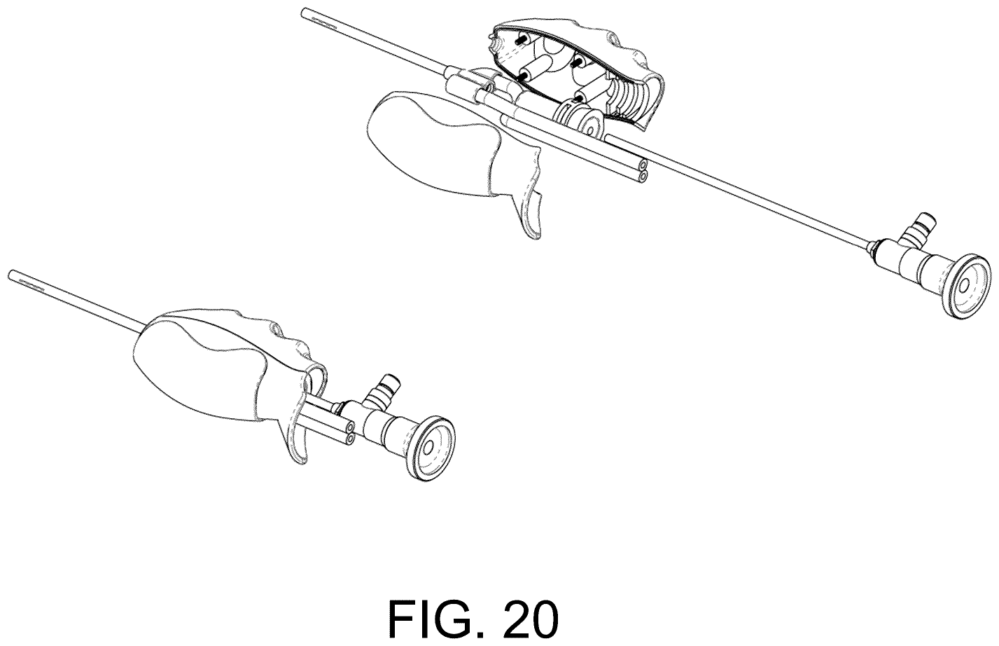

In various further aspects, FIGS. 20-22 illustrate yet another example embodiment of a device in accordance with the present invention. The device in this illustrated embodiment includes a large hand piece and is arranged to provide both suction and irrigation in the operative field.

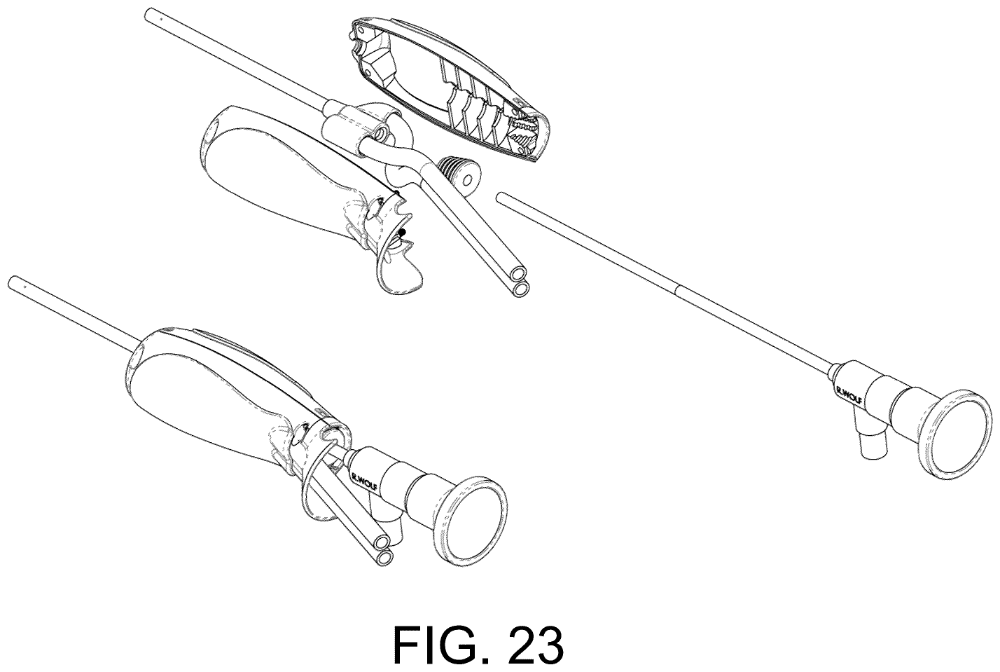

In various further aspects, FIGS. 23-25 illustrate yet another example embodiment of a device in accordance with the present invention. The device in this illustrated embodiment includes a large hand piece and is arranged to provide both suction and irrigation in the operative field.

Also disclosed herein are methods of making and using the disclosed devices, apparatuses, and systems. For example, in another exemplary aspect, the present disclosure provides a method for using a disclosed scope device and system in medical procedures. For example, the scope system may be in treating disorders of the ear, nose, and throat.

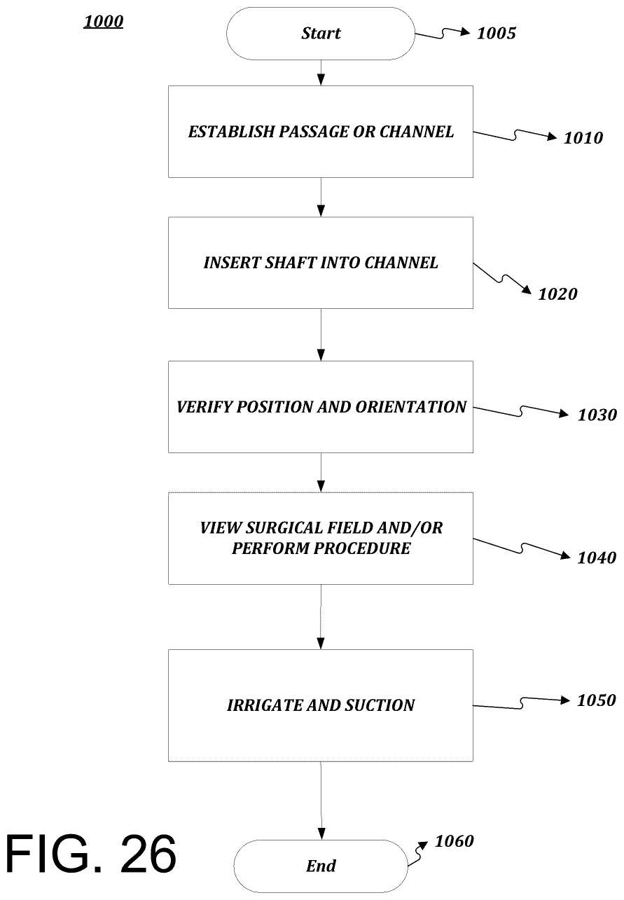

FIG. 26 is a flow chart setting forth various stages involved in a method 1000 consistent with an embodiment of the disclosure for using the disclosed scope apparatuses, devices and systems. Method 1000 may be implemented using any disclosed apparatus or device, at least in part, for example, apparatus 100 or 800, as described in more detail with respect to FIGS. 1-25.

Furthermore, although stages are disclosed with reference to apparatus 100, it should be understood that other disclosed device embodiments may enable the operation of method 1000, including, but not limited to, other device mechanisms, mechanical components, use indication (e.g., ear, nose, throat etc.), patient conditions, and the like. Further still, although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. It will be appreciated that scope devices and systems as presented herein may be useful in a variety of medical procedures, e.g., surgery, scope procedure, and the like. One advantage of the present invention is that it may be suitable for use with existing surgical and medical techniques requiring a scope. In relevant part, using the scope according to the present disclosure may involve a physician or surgeon inserting the appropriate portion of the scope in the patient. A next step may involve the surgeon inserting the shaft into the patient. Once properly positioned in the desired location, the surgeon may then adjust the length of the scope. The surgeon may continue to fit to diameter of scope until the desired visualization of the surgical field has been achieved. The surgeon may then Irrigate and suction to maintain clear visibility and a clean surgical field. In some aspects, a wedge may accommodate multiple scope diameters and helps with rotation of surgical field.

In further aspects, method 1000 may begin at starting block 1005 and proceed to stage 1010, where a passage or channel is identified or established in a patient. A passage or channel may be existing channel or may established by any method currently or prospectively utilized. However, one skilled in the art would recognize that the method of establishing a channel may vary according to many factors such as the type of tissue, the location of the desired channel, or other factors specific to the variance associated with different patients' anatomy or specific needs and surgeon's preference.

From stage 1010, where the passage or channel is identified or established, method 1000 may proceed to stage 1020 where the shaft may be inserted into the passage or established channel. The device may be characterized by, but not limited to, the various embodiments depicted above in FIGS. 1-25 and/or may include various scope characteristics with different handpieces for holding and orienting the device. One skilled in the art would recognize that the insertion of the device may vary according to factors specific to the variance associated with different patients' anatomy or specific types of tissue or needs as required to successfully complete the task.

From stage 1020, where the device shaft is inserted, method 1000 may proceed to stage 1030, where proper positioning and orientation may be verified using, for example, an integrated camera, visual cues and/or markers and radiographs. For example, markers may provide useful information regarding the position of the scope in the channel, but not limited to, the position or length of the scope in the channel, the length and width of the scope once inside the channel or shaft, the angle of entry into the channel, and/or the depth of the scope in the channel. In further aspects, a wedge can accommodate multiple scope diameters and helps with rotation of surgical field.

From stage 1030, where the proper positioning and orientation may be confirmed, method 1000 may proceed to stage 1040, where the medical procedure may begin. For example, the surgical field may be viewed, and the procedure commenced.

From stage 1040, where the medical procedure is performed, method 1000 may proceed to stage 1050, where, if needed, the area may be irrigated and suctioned with a saline solution, an irrigant, or otherwise to maintain clear visibility and a clean surgical field. Alternatively, or in addition to stage 1050, the channel or area may be irrigated and suctioned with a saline solution or an irrigant in a prior or subsequent step.

From stage 1050, where the irrigation and suction is performed, method 1000 may end at stage 1060, where, the device can be removed from the patient.

In further aspects, the present disclosure provides a method for providing continuous suction and irrigation during an endoscopic procedure. In still further aspects, the method can comprise one or more of following steps: providing a surgical hand piece apparatus with an adjustable sheath, a combined suction and irrigation port, and a mechanical foot pedal assembly; inserting an endoscope into the adjustable sheath of the surgical hand piece apparatus; connecting the combined suction and irrigation port to a source of irrigation fluid and a suction device; actuating a first pedal of the mechanical foot pedal assembly to control the flow of irrigation fluid through the combined suction and irrigation port and the adjustable sheath to the endoscope; and/or actuating a second pedal of the mechanical foot pedal assembly to control the suction through the combined suction and irrigation port and the adjustable sheath from the endoscope. In yet further aspects, the method can further comprise one or more of following steps: adjusting the length of the adjustable sheath to accommodate the endoscope of varying lengths prior to the inserting step; wherein the actuating steps for the first and second pedals are performed using color-coded pedals for easy identification of the irrigation and suction controls; supporting the surgical hand piece apparatus on the thenar eminence of a user's hand during the inserting step; wherein the providing step includes selecting a surgical hand piece apparatus with an ergonomic hand grip portion designed to reduce carpal tunnel pressure and fatigue; maintaining the mechanical foot pedal assembly in a default position that closes the spring-loaded pinch valves when the pedals are not engaged; and/or wherein the connecting step includes using a single Luer lock or barbed fitting to establish the connection to the source of irrigation fluid and the suction device.

In further aspects, the present disclosure provides a method for maintaining visibility and cleanliness of an endoscopic lens during a surgical procedure. In still further aspects, the method can comprise one or more of following steps: providing a surgical hand piece with an integrated adjustable sheath and a combined suction and irrigation port; inserting an endoscope through the adjustable sheath; connecting a source of irrigation fluid and a suction device to the combined suction and irrigation port; actuating a mechanical foot pedal assembly to control the flow of irrigation fluid to the endoscope; and actuating the mechanical foot pedal assembly to control suction from the operative field through the endoscope. In yet further aspects, the method can further comprise one or more of following steps or features: wherein the adjustable sheath is configured to accommodate endoscopes of various diameters; wherein the irrigation fluid includes an antibiotic solution; wherein the irrigation fluid is warmed to facilitate paradoxical vasoconstriction during the surgical procedure; wherein the mechanical foot pedal assembly includes spring-loaded pinch valves for controlling the flow of irrigation fluid and suction; wherein the mechanical foot pedal assembly is non-electronic; wherein the surgical hand piece includes an ergonomic design to reduce fatigue during the surgical procedure; adjusting the effective length of the adjustable sheath to match the length of the endoscope; wherein the combined suction and irrigation port utilizes a single Luer lock or barbed fitting for both suction and irrigation; wherein the actuating step for controlling irrigation fluid is performed independently from the actuating step for controlling suction; cleaning the lens of the endoscope with the irrigation fluid without removing the endoscope from the operative field; wherein the suction is used to evacuate blood and debris from the operative field; wherein the mechanical foot pedal assembly includes color-coded pedals for distinguishing between irrigation and suction controls; wherein the surgical hand piece is supported on the thenar eminence of the user's hand during the procedure; wherein the surgical hand piece includes a soft grip material over a hard molded housing for comfort; wherein the surgical hand piece is optimized for use during sinuplasty procedures; wherein the mechanical foot pedal assembly provides continuous control over the flow of fluids; wherein the surgical hand piece accommodates multiple scopes from various manufacturers; wherein the surgical hand piece includes a sheath with an internal sealing o-ring to maintain a fluid-tight seal around the endoscope; wherein the surgical hand piece includes a flow valve assembly operative to direct fluid flow through the adjustable sheath; wherein the surgical hand piece is designed to provide carpal tunnel fatigue relief; wherein the mechanical foot pedal assembly is configured to operate the spring-loaded pinch valves independently for suction and irrigation; the surgical hand piece and the adjustable sheath are configured to allow for quick disassembly for cleaning and sterilization; wherein the surgical hand piece includes a rigid structure with a soft rubber overmold; wherein the surgical hand piece is universally compatible with endoscopes from multiple manufacturers; wherein the surgical hand piece includes a thenar rest to allow support of the hand piece on the thumb web; wherein the surgical hand piece includes a soft grip cover that is overmolded; wherein the surgical hand piece includes an adjustable length sheath with a designed Luer lock; wherein the surgical hand piece includes a suction/irrigation combined port attached to the hand piece; and/or wherein the surgical hand piece includes a mechanical foot pedal with pedals for suction and irrigation, and the two valves are color-coded for easy application by the operating room staff.

In further aspects, the present disclosure provides a method for facilitating an endoscopic procedure with an adjustable sheath. In still further aspects, the method may comprise one or more of the following steps or features: providing a surgical hand piece with an integrated adjustable sheath capable of accommodating various endoscope lengths; inserting an endoscope into the adjustable sheath until a desired length is achieved; securing the endoscope within the adjustable sheath using a securement feature; directing irrigation fluid through a fluid channel in the adjustable sheath to the distal end of the endoscope; and/or facilitating suction from the proximal end of the endoscope through the fluid channel in the adjustable sheath.

In further aspects, the present disclosure provides a method for controlling fluid flow during an endoscopic procedure. In still further aspects, the method can comprise one or more of following steps or features: providing a surgical hand piece with an integrated adjustable sheath and a combined suction and irrigation port; adjusting a valve mechanism within the adjustable sheath to regulate irrigation fluid flow to the endoscope; actuating a control interface on the surgical hand piece to modify the suction force through the endoscope; and/or maintaining continuous fluid flow and suction during the endoscopic procedure through user manipulation of the control interface.

In further aspects, the present disclosure provides a method for capturing and transmitting endoscopic images. In still further aspects, the method can comprise one or more of following steps or features: capturing visual data from an operative field using an electronic image sensor within an endoscope; processing the visual data into electronic image signals with a data processing unit; transmitting the electronic image signals to an external processing system via a transmission module; and/or powering the electronic image sensor and data processing unit with a power supply mechanism.

In further aspects, the present disclosure provides a method for enhancing visualization during an endoscopic procedure. In still further aspects, the method can comprise one or more of following steps or features: inserting an endoscope with an integrated camera into an adjustable sheath; delivering an irrigation fluid through the adjustable sheath to maintain cleanliness of the endoscope lens; capturing images of the operative field with the integrated camera; and/or transmitting the captured images to an external display via a connection interface.

In further aspects, the present disclosure provides a method for customizing a surgical hand piece for endoscopic procedures. In still further aspects, the method can comprise one or more of following steps or features: selecting an ergonomic grip module from a set of interchangeable modules based on a user's hand size and preference; attaching the selected ergonomic grip module to a main body of the surgical hand piece using a quick-connect mechanism; choosing a fluid control module from a set of interchangeable modules to match the procedural irrigation and suction needs; connecting the chosen fluid control module to the main body; and securing a detachable adjustable sheath to the main body to accommodate the endoscope being used.

In further aspects, the present disclosure provides a method for sterilizing an endoscopic apparatus, comprising: disassembling the endoscopic apparatus into components suitable for sterilization; subjecting the components to a high-temperature autoclave sterilization process; performing chemical disinfection on components sensitive to high temperatures; reassembling the sterilized components into a functional endoscopic apparatus; and/or verifying the sterilization efficacy through visual indicators on the components.

In further aspects, the present disclosure provides a method for managing irrigation and suction during endoscopic procedures, comprising: providing an endoscopic apparatus with an integrated fluid control system; delivering irrigation fluid to the operative field through a dedicated irrigation channel; removing fluids from the operative field using a dedicated suction channel; independently controlling the flow rates of irrigation and suction via a valve assembly; and/or collecting the removed fluids in a waste collection container.

In further aspects, the present disclosure provides a method for enhancing ergonomic usability of an endoscopic apparatus, comprising: providing a surgical hand piece with a design based on human factors engineering; adjusting the weight distribution within the hand piece to achieve ergonomic balance; customizing the grip texture and support features to fit the user's hand; positioning the user interface for easy access to apparatus controls; and utilizing ergonomic visual indicators to guide the user in making adjustments for optimal comfort and usability.

In further aspects, the present disclosure provides a method for performing a surgical procedure using a disclosed endoscopic apparatus with integrated fluid control and ergonomic design, comprising: positioning the surgical hand piece of the endoscopic apparatus to access the operative field; activating the fluid control system to deliver irrigation fluid to the operative field and suction fluids from the operative field; manipulating the endoscope to visualize the surgical site through a remote visualization unit; adjusting the ergonomic features of the hand piece to conform to the user's hand for comfortable operation; and/or operating the endoscopic apparatus controls to perform the surgical procedure while maintaining ergonomic posture.

In further aspects, the present disclosure provides a method for configuring a disclosed endoscopic apparatus for specific surgical procedures, comprising: selecting an endoscope module from a set of interchangeable modules based on the surgical procedure; attaching the selected endoscope module to a base surgical hand piece using a universal connector; customizing a fluid control system with modular components to meet the irrigation and suction needs of the procedure; choosing an ergonomic grip from a selection of grips to fit the user's hand and procedure requirements; and/or programming a user interface module with control settings specific to the surgical procedure.

In further aspects, the present disclosure provides a method for integrating an endoscopic apparatus with various surgical environments and equipment, comprising: connecting the apparatus to a surgical monitor using a universal interface; adapting the power supply of the apparatus to match the surgical environment's electrical standards; calibrating the apparatus based on readings from surgical environment sensors; synchronizing the apparatus with hospital information systems for seamless data exchange; and/or utilizing a sterilizable docking station for charging and data synchronization between surgeries.

In further aspects, the present disclosure provides a method for remote visualization and control during endoscopic procedures, comprising: wirelessly transmitting visual data from an endoscope to a remote visualization unit; displaying the received visual data on the remote visualization unit; remotely adjusting endoscopic functions via a control interface on the endoscope; and synchronizing the endoscope and the remote visualization unit to maintain a secure wireless connection.

Also disclosed herein are kits comprising the disclosed scope devices and systems. For example, in an exemplary aspect, the present disclosure provides a scope kit comprising: a disclosed scope device; and instructions for using the device in connection with a medical procedure. In further aspects, the instruction and/or method may comprise any medical procedure and/or method step. In still further aspects, the method and/or method step may comprise inserting a disclosed scope device in a patient, such as a human or mammal.

The present disclosure includes at least the following aspects: 1. An endoscopic apparatus comprising: a body portion having an inner cavity operative to engage and retain an endoscope; a first tubing portion having a first distal end and a second distal end arranged in the inner cavity; a fluid port operative to engage the first distal end of the first tubing portion; a flow assembly operative to engage the second distal end of the first tubing portion and a portion of the endoscope; and a hand grip portion arranged on the body portion.

Aspect 1: A surgical hand piece apparatus for endoscopic procedures, comprising: a body portion configured to receive and retain an endoscope; an adjustable sheath coupled to the body portion, wherein the adjustable sheath is configured to accommodate endoscopes of varying lengths; a combined suction and irrigation port integrated into the body portion; a mechanical foot pedal assembly communicatively connected to the combined suction and irrigation port, the mechanical foot pedal assembly including spring-loaded pinch valves for controlling fluid flow; and an ergonomic hand grip portion formed on the body portion, the ergonomic hand grip portion including a soft overmolded material designed to reduce carpal tunnel pressure and fatigue during use. Aspect 2: The apparatus of claim 43, wherein the adjustable sheath includes a Luer lock mechanism for securing the endoscope. Aspect 3:

The apparatus of claim 43, wherein the combined suction and irrigation port comprises a single Luer lock or barbed fitting. Aspect 4: The apparatus of claim 43, wherein the mechanical foot pedal assembly is non-electronic. Aspect 5: The apparatus of claim 43, wherein the spring-loaded pinch valves are color-coded for easy identification of suction and irrigation controls. Aspect 6: The apparatus of claim 43, wherein the ergonomic hand grip portion is designed to support the thenar eminence of a user's hand. Aspect 7: The apparatus of claim 43, wherein the body portion further comprises a rigid structure with a soft rubber overmold. Aspect 8: The apparatus of claim 43, wherein the adjustable sheath is configured to provide a fluid flow path for both irrigation and suction fluids. Aspect 9: The apparatus of claim 43, wherein the mechanical foot pedal assembly includes a heavy base for stability during operation. Aspect 10: The apparatus of claim 43, wherein the adjustable sheath is configured to translate relative to the body portion to accommodate different endoscope models. Aspect 11: The apparatus of claim 43, wherein the ergonomic hand grip portion includes a thenar rest to allow support of the hand piece on the thumb web. Aspect 12: The apparatus of claim 43, wherein the mechanical foot pedal assembly is configured to return to a default position that closes the spring-loaded pinch valves when not engaged.

Aspect 13: The apparatus of claim 43, wherein the combined suction and irrigation port is configured to allow continuous flow of fluids without interruption.

Aspect 14: The apparatus of claim 43, wherein the body portion is configured to be universally compatible with multiple endoscope manufacturers.

Aspect 15: The apparatus of claim 43, wherein the ergonomic hand grip portion is optimized for use during sinuplasty procedures.

Aspect 16: The apparatus of claim 43, wherein the mechanical foot pedal assembly provides continuous control over the flow of fluids.

Aspect 17: The apparatus of claim 43, wherein the adjustable sheath includes an internal sealing o-ring to maintain a fluid-tight seal around the endoscope.

Aspect 18: The apparatus of claim 43, wherein the body portion includes a flow valve assembly operative to direct fluid flow through the adjustable sheath.

Aspect 19: The apparatus of claim 43, wherein the ergonomic hand grip portion is designed to provide carpal tunnel fatigue relief.

Aspect 20: The apparatus of claim 43, wherein the mechanical foot pedal assembly is configured to operate the spring-loaded pinch valves independently for suction and irrigation.

Aspect 21: The apparatus of claim 43, wherein the body portion and the adjustable sheath are configured to allow for quick disassembly for cleaning and sterilization.

Aspect 22: A surgical hand piece apparatus for endoscopic procedures, comprising: a body configured to accommodate an endoscope; an ergonomic grip portion designed to conform to the contours of a user's hand; an integrated adjustable sheath for encasing a portion of the endoscope and allowing length adjustment; a combined suction and irrigation port connected to the body for providing fluid flow and suction through the endoscope; and a thenar rest positioned to support the hand piece on the user's thenar eminence during use.

Aspect 24: The apparatus of claim 43, wherein the ergonomic grip portion includes a soft grip material over a hard molded housing to enhance comfort and reduce hand fatigue.

Aspect 25: The apparatus of claim 43, wherein the ergonomic grip portion is designed to assist with the relief of carpal tunnel pressure during prolonged surgical procedures.

Aspect 26: The apparatus of claim 43, wherein the integrated adjustable sheath includes a Luer lock mechanism for securing the endoscope at various lengths.

Aspect 27: The apparatus of claim 43, wherein the combined suction and irrigation port is designed to maintain a continuous flow of irrigation fluid and suction without interruption.

Aspect 28: The apparatus of claim 43, wherein the thenar rest is contoured to provide stability and control of the hand piece during endoscopic maneuvers.

Aspect 29: The apparatus of claim 43, further comprising a fluid channel within the adjustable sheath that is optimized to prevent clogging and ensure clear visibility through the endoscope.

Aspect 30: The apparatus of claim 43, wherein the body of the hand piece is constructed from materials compatible with sterilization processes used in medical facilities.

Aspect 31: The apparatus of claim 43, wherein the ergonomic grip portion includes a textured surface to improve grip and prevent slippage.

Aspect 32: The apparatus of claim 43, wherein the body of the hand piece is designed to be lightweight to minimize user fatigue during extended use.

Aspect 33: The apparatus of claim 43, wherein the ergonomic grip portion is available in multiple sizes to accommodate different hand sizes of users.

Aspect 34: The apparatus of claim 43, wherein the thenar rest includes a cushioned pad for added comfort during use.

Aspect 35: The apparatus of claim 43, wherein the body of the hand piece includes visual indicators for alignment with the endoscope.

Aspect 36: The apparatus of claim 43, wherein the integrated adjustable sheath is detachable for easy cleaning and replacement.

Aspect 37: The apparatus of claim 43, wherein the ergonomic grip portion is designed to allow for multiple user adaptations, including grip size and shape.

Aspect 38: The apparatus of claim 43, wherein the body of the hand piece includes an integrated channel for passing additional surgical instruments alongside the endoscope.

Aspect 39: The apparatus of claim 43, wherein the combined suction and irrigation port includes a single Luer lock or barbed fitting for streamlined connection to fluid sources.

Aspect 40: The apparatus of claim 43, wherein the ergonomic grip portion is designed to distribute pressure evenly across the user's hand to prevent pressure points.

Aspect 41: The apparatus of claim 43, wherein the body of the hand piece is designed to facilitate a neutral wrist position to reduce the risk of repetitive strain injuries.

Aspect 42: The apparatus of claim 43, wherein the body of the hand piece includes a modular design for customization and adaptability to various endoscopic procedures.

Aspect 43: A fluid control apparatus for an endoscopic surgical hand piece, comprising: [0145] an integrated adjustable sheath designed to accommodate an endoscope and provide a fluid pathway for irrigation and suction; a combined suction and irrigation port operatively connected to the adjustable sheath; a valve mechanism within the adjustable sheath for regulating the flow of irrigation fluid and suction; and a control interface accessible on the surgical hand piece for user manipulation of the valve mechanism. Aspect 45: The apparatus of claim 43, wherein the valve mechanism includes a spring-loaded pinch valve for each of the irrigation and suction pathways.

Aspect 46: The apparatus of claim 43, wherein the combined suction and irrigation port includes a single connection point for both irrigation fluid supply and suction evacuation.

Aspect 47: The apparatus of claim 43, wherein the control interface comprises a mechanical actuator linked to the valve mechanism for precise flow control.

Aspect 48: The apparatus of claim 43, wherein the adjustable sheath includes a transparent section for visual monitoring of fluid flow.

Aspect 49: The apparatus of claim 43, wherein the valve mechanism is configured to maintain a continuous and uninterrupted flow when in an open position.

Aspect 50: The apparatus of claim 43, wherein the control interface includes ergonomic features to facilitate operation with minimal hand movement.

Aspect 51: The apparatus of claim 43, wherein the valve mechanism is replaceable for maintenance and longevity of the apparatus.

Aspect 52: The apparatus of claim 43, wherein the combined suction and irrigation port is designed to prevent backflow and cross-contamination between the pathways.

Aspect 53: The apparatus of claim 43, wherein the adjustable sheath and the valve mechanism are constructed from materials resistant to common sterilization processes.

Aspect 54: An endoscopic visualization apparatus, comprising: an endoscope with an integrated camera for capturing images within a patient's body; an adjustable sheath configured to encase a portion of the endoscope and provide a clear fluid pathway for maintaining lens cleanliness; a fluid control system integrated into the adjustable sheath for delivering an irrigation fluid to the distal end of the endoscope to enhance image clarity; and a connection interface for transmitting captured images from the integrated camera to an external display.

Aspect 56: The apparatus of claim 43, wherein the adjustable sheath includes a transparent section for direct visual inspection of the endoscope lens.

Aspect 57: The apparatus of claim 43, wherein the fluid control system comprises a valve mechanism for regulating the flow of the irrigation fluid to prevent lens obstruction.

Aspect 58: The apparatus of claim 43, wherein the connection interface includes a high-definition video output compatible with various external display technologies.

Aspect 59: The apparatus of claim 43, wherein the integrated camera is capable of capturing images in low-light conditions within the patient's body.

Aspect 60: The apparatus of claim 43, wherein the fluid control system is designed to deliver a steady stream of irrigation fluid without disrupting the captured images.

Aspect 61: The apparatus of claim 43, wherein the adjustable sheath is designed to minimize glare and reflections that may interfere with imaging quality.

Aspect 62: The apparatus of claim 43, wherein the integrated camera includes a zoom function for detailed visualization of the operative field.

Aspect 63: The apparatus of claim 43, wherein the connection interface supports real-time image processing and enhancement features.

Aspect 64: The apparatus of claim 43, wherein the adjustable sheath and fluid control system are autoclavable for sterilization without damaging the integrated camera.

Aspect 65: A modular surgical hand piece apparatus for endoscopic procedures, comprising: a main body with a universal interface for attaching various ergonomic grip modules; a selection of interchangeable ergonomic grip modules designed to accommodate different hand sizes and preferences; a detachable adjustable sheath component for encasing an endoscope, with variable length settings; a set of interchangeable fluid control modules for customizing irrigation and suction capabilities; and a quick-connect mechanism for attaching and detaching the ergonomic grip modules and fluid control modules to the main body.

Aspect 67: The modular surgical hand piece apparatus of claim 43, wherein each ergonomic grip module includes a distinct texture or material to provide various tactile experiences.

Aspect 68: The apparatus of claim 43, wherein the detachable adjustable sheath component includes a range of diameters to fit different endoscope models.

Aspect 69: The apparatus of claim 43, wherein the fluid control modules include options for manual or automated flow regulation.

Aspect 70: The apparatus of claim 43, wherein the quick-connect mechanism comprises a locking collar that ensures secure attachment while allowing for easy module exchange.

Aspect 71: The apparatus of claim 43, further comprising a storage case for organizing and maintaining the selection of interchangeable modules.

Aspect 72: The apparatus of claim 43, wherein the main body includes an integrated electronic interface for connecting to endoscope imaging systems.

Aspect 73: The apparatus of claim 43, wherein the ergonomic grip modules are designed to reduce the risk of repetitive strain injuries through ergonomic assessment.

Aspect 74: The apparatus of claim 43, wherein the fluid control modules are color-coded to facilitate identification and selection based on procedural requirements.

Aspect 75: The apparatus of claim 43, wherein the main body is constructed from a material compatible with high-level disinfection and sterilization processes.

Aspect 76: An endoscopic image capture and transmission apparatus, comprising: an endoscope equipped with an electronic image sensor for capturing visual data; a data processing unit integrated within the endoscope for converting visual data into electronic image signals; a transmission module for sending electronic image signals to an external processing system; and a power supply mechanism for providing electrical energy to the electronic image sensor and data processing unit.

Aspect 78: The apparatus of claim 43, wherein the data processing unit includes an image signal processor for enhancing image quality.

Aspect 79: The apparatus of claim 43, wherein the transmission module includes a wireless communication interface for remote image signal transmission.

Aspect 80: The apparatus of claim 43, wherein the power supply mechanism comprises a rechargeable battery integrated into the endoscope handle.

Aspect 81: The apparatus of claim 43, wherein the electronic image sensor is capable of capturing high-resolution images suitable for diagnostic purposes.

Aspect 82: The apparatus of claim 43, wherein the transmission module is compatible with standard medical image storage and communication protocols.

Aspect 83: The apparatus of claim 43, wherein the data processing unit includes real-time image stabilization features to compensate for motion during the endoscopic procedure.

Aspect 84: The apparatus of claim 43, wherein the power supply mechanism includes a low-battery indicator to alert the user of the need for recharging or replacement.

Aspect 85: The apparatus of claim 43, wherein the transmission module supports high-bandwidth data transfer to minimize latency in image display.

Aspect 86: The apparatus of claim 43, wherein the electronic image sensor includes a light-sensitive chip for capturing images in variable lighting conditions within the patient's body.

Aspect 87: A wireless communication and connectivity apparatus for endoscopic procedures, comprising: an endoscope with an integrated wireless transmitter for sending visual data; a remote visualization unit capable of receiving and displaying the visual data transmitted by the endoscope; a control interface on the endoscope for wireless operation of endoscopic functions; and a synchronization module for establishing and maintaining a secure wireless connection between the endoscope and the remote visualization unit. Aspect 89: The wireless communication and connectivity apparatus of claim 43, wherein the integrated wireless transmitter operates on a frequency band reserved for medical devices to minimize interference.

Aspect 90: The apparatus of claim 43, wherein the remote visualization unit includes a high-definition display for detailed image analysis.

Aspect 91: The apparatus of claim 43, wherein the control interface allows for remote adjustment of endoscopic settings such as light intensity and image capture modes.

Aspect 92: The apparatus of claim 43, wherein the synchronization module utilizes encryption protocols to ensure the privacy and security of transmitted data.

Aspect 93: The apparatus of claim 43, further comprising a wireless receiver integrated into the endoscope for receiving control signals from the remote visualization unit.

Aspect 94: The apparatus of claim 43, wherein the remote visualization unit is equipped with touch-screen capabilities for intuitive control and navigation.

Aspect 95: The apparatus of claim 43, wherein the endoscope includes a rechargeable battery with wireless charging capabilities for continuous operation.

Aspect 96: The apparatus of claim 43, wherein the synchronization module supports multiple wireless communication standards for compatibility with various external devices.

Aspect 97: The apparatus of claim 43, wherein the control interface includes haptic feedback to confirm successful operation of remote commands.

Aspect 98: A sterilizable endoscopic apparatus, comprising: a surgical hand piece constructed from materials resistant to high-temperature sterilization processes; an endoscope with components and assemblies designed to withstand chemical disinfection; a detachable sheath made from a biocompatible material suitable for autoclave sterilization; and a fluid control system with components that maintain functionality after exposure to sterilizing agents.

Aspect 100: The apparatus of claim 43, wherein the surgical hand piece materials include medical-grade stainless steel and high-temperature polymers.

Aspect 101: The apparatus of claim 43, wherein the endoscope components are sealed to prevent damage from liquid disinfectants.

Aspect 102: The apparatus of claim 43, wherein the detachable sheath is coated with an antimicrobial layer to enhance sterilization efficacy.

Aspect 103: The apparatus of claim 43, wherein the fluid control system includes valves and seals made from silicone or other elastomers compatible with sterilization processes.

Aspect 104: The apparatus of claim 43, further comprising a wireless communication module with encapsulation to protect electronic components during sterilization.

Aspect 105: The apparatus of claim 43, wherein the endoscope includes a removable camera module designed for separate sterilization.

Aspect 106: The apparatus of claim 43, wherein the materials used for the surgical hand piece and detachable sheath are certified for repeated sterilization cycles without degradation.

Aspect 107: The apparatus of claim 43, wherein the fluid control system is modular, allowing for disassembly and individual sterilization of components.

Aspect 108: The apparatus of claim 43, wherein the apparatus includes indicators that change color or appearance to confirm successful sterilization.

Aspect 109: An endoscopic apparatus with integrated fluid control, comprising: a surgical hand piece with an embedded fluid control system for managing irrigation and suction during endoscopic procedures; a dual-channel tubing system within the hand piece, one for delivering irrigation fluid to the operative field and another for suction removal of fluids; a valve assembly operable to independently control the flow rate of irrigation and suction; a fluid reservoir connectable to the irrigation channel for supplying the irrigation fluid; and a waste collection container connectable to the suction channel for collecting fluids removed from the operative field.

Aspect 111: The apparatus of claim 43, wherein the valve assembly includes adjustable controls for fine-tuning the flow rates of irrigation and suction in real-time.

Aspect 112: The endoscopic apparatus of claim 43, wherein the fluid reservoir is designed to maintain a sterile environment for the irrigation fluid.

Aspect 113: The apparatus of claim 43, wherein the waste collection container includes a filter mechanism to separate debris from the suctioned fluids.

Aspect 114: The apparatus of claim 43, further comprising a pressure regulation system to ensure consistent irrigation flow and suction vacuum levels.

Aspect 115: The apparatus of claim 43, wherein the dual-channel tubing system is made from a flexible, biocompatible material that resists kinking and blockage.

Aspect 116: The apparatus of claim 43, wherein the surgical hand piece includes a user interface with indicators for monitoring fluid levels and flow rates.

Aspect 117: The apparatus of claim 43, wherein the fluid control system is capable of simultaneous irrigation and suction without cross-contamination.

Aspect 118: The apparatus of claim 43, wherein the valve assembly is operable via afoot pedal mechanism, allowing hands-free control by the user.

Aspect 119: The apparatus of claim 43, wherein the fluid reservoir and waste collection container are easily detachable for quick replacement during procedures.

Aspect 120: An ergonomically designed endoscopic apparatus, comprising: a surgical hand piece with a contoured grip conforming to human hand ergonomics to reduce fatigue during prolonged use; [0214] a weight distribution system within the hand piece to balance the apparatus and minimize strain on the user's wrist and arm; a textured surface on the grip areas to enhance grip stability and prevent slippage; adjustable support features to accommodate various hand sizes and grip styles; and a user interface positioned to allow intuitive access to controls without requiring significant hand movement.

Aspect 122: The apparatus of claim 43, wherein the contoured grip includes a padded overlay for additional comfort.

Aspect 123: The apparatus of claim 43, wherein the weight distribution system is adjustable to adapt to different endoscope weights and lengths.

Aspect 124: The apparatus of claim 43, wherein the textured surface comprises a non-slip silicone or rubber material.

Aspect 125: The apparatus of claim 43, wherein the adjustable support features include a thumb rest and finger grooves.

Aspect 126: The apparatus of claim 43, wherein the user interface includes touch-sensitive controls that respond to light pressure.

Aspect 127: The apparatus of claim 43, further comprising a detachable wrist support to maintain the hand in a neutral position.

Aspect 128: The apparatus of claim 43, wherein the hand piece is designed to reduce pressure points and prevent carpal tunnel syndrome.

Aspect 129: The apparatus of claim 43, wherein the grip areas are thermally insulated to maintain a comfortable temperature during use.

Aspect 130: The apparatus of claim 43, wherein the hand piece includes visual indicators for ergonomic adjustments to guide the user to an optimal grip configuration.

Aspect 132: A modular endoscopic apparatus, comprising: a base surgical hand piece with a universal connector for attaching various endoscopic tools; a set of interchangeable endoscope modules, each designed for specific surgical procedures; a customizable fluid control system with modular components for irrigation and suction tailored to procedure-specific requirements; a selection of ergonomic grips of varying sizes and shapes to accommodate user preference and procedure demands; and a user interface module that can be configured with procedure-specific control settings.

Aspect 134: The apparatus of claim 43, wherein the universal connector includes a quick-release mechanism for rapid tool exchange.

Aspect 135: The apparatus of claim 43, wherein each interchangeable endoscope module is optimized for a particular field of view and depth of focus.

Aspect 136: The apparatus of claim 43, wherein the customizable fluid control system includes interchangeable valves and pumps for different flow rate requirements.

Aspect 137: The apparatus of claim 43, wherein the ergonomic grips feature a tool-free attachment system for easy customization.

Aspect 138: The apparatus of claim 43, wherein the user interface module includes a touch screen that can be programmed with custom control profiles.

Aspect 139: The apparatus of claim 43, further comprising a selection of sheaths with different diameters and lengths for various endoscope modules.

Aspect 140: The apparatus of claim 43, wherein the base surgical hand piece includes an integrated power supply module that can be swapped for different battery capacities.

Aspect 141: The apparatus of claim 43, wherein the fluid control system modules are color-coded for easy identification and matching with corresponding surgical procedures.

Aspect 142: The apparatus of claim 43, wherein the user interface module is capable of wirelessly syncing with a central database to update control settings and software.

Aspect 143: The apparatus of claim 43, wherein the universal interface includes multiple video output formats to ensure compatibility with high-definition and 3D surgical monitors.

Aspect 144: The apparatus of claim 43, wherein the adaptable power supply features an automatic voltage adjustment system for international use.

Aspect 145: The apparatus of claim 43, wherein the surgical environment sensors detect lighting conditions, temperature, and humidity to optimize the apparatus performance.

Aspect 146: The apparatus of claim 43, wherein the communication module supports wireless and wired connectivity protocols, including Bluetooth, Wi-Fi, and Ethernet.

Aspect 147: The apparatus of claim 43, wherein the sterilizable docking station includes RFID or barcode scanning for tracking and inventory management.

Aspect 148: The apparatus of claim 43, further comprising a modular accessory kit with various endoscopic tools for different surgical specialties.

Aspect 149: The apparatus of claim 43, wherein the communication module is capable of receiving software updates and configuration changes remotely.

Aspect 150: The apparatus of claim 43, wherein the universal interface is designed to connect with robotic surgical systems for enhanced control during minimally invasive procedures.

Aspect 151: The apparatus of claim 43, wherein the docking station includes a user authentication system to ensure secure access to the apparatus.

Aspect 152: An adjustable sheath apparatus for use with a surgical hand piece in endoscopic procedures, comprising: a sheath body configured to encase a portion of an endoscope; an adjustment mechanism integrated into the sheath body for altering the effective length of the sheath to accommodate endoscopes of varying lengths; a fluid channel within the sheath body designed to direct irrigation fluid towards the distal end of the endoscope and to facilitate suction from the proximal end; a securement feature for maintaining a fluid-tight connection between the sheath body and the endoscope; and an attachment interface for removably coupling the adjustable sheath apparatus to the surgical hand piece.

Aspect 153: The adjustable sheath apparatus of claim 43, wherein the adjustment mechanism includes a sliding collar that locks into place at various positions along the sheath body.

Aspect 154: The adjustable sheath apparatus of claim 43, wherein the securement feature comprises a Luer lock mechanism compatible with standard endoscope designs.

Aspect 155: The adjustable sheath apparatus of claim 43, wherein the fluid channel is configured to maintain separation between the irrigation fluid and the suctioned materials.

Aspect 156: The adjustable sheath apparatus of claim 43, wherein the attachment interface includes a quick-release mechanism for rapid detachment from the surgical hand piece.

Aspect 157: The adjustable sheath apparatus of claim 43, wherein the sheath body is constructed from a biocompatible and corrosion-resistant material suitable for medical use.

Aspect 158: The adjustable sheath apparatus of claim 43, wherein the sheath body includes a transparent section for visual monitoring of the fluid channel.

Aspect 159: The adjustable sheath apparatus of claim 43, wherein the adjustment mechanism is operable with a single hand to facilitate ease of use during surgical procedures.

Aspect 160: The adjustable sheath apparatus of claim 43, wherein the securement feature includes an internal sealing element, such as an o-ring, to enhance the fluid-tight connection.

Aspect 161: The adjustable sheath apparatus of claim 43, wherein the sheath body is designed to provide structural support to the endoscope while allowing flexibility for maneuvering.

Aspect 162: The adjustable sheath apparatus of claim 43, wherein the attachment interface is compatible with multiple models of surgical hand pieces.

Aspect 163: The adjustable sheath apparatus of claim 43, wherein the sheath body includes graduated markings to indicate the adjusted length of the sheath.

Aspect 164: The adjustable sheath apparatus of claim 43, wherein the sheath body is autoclavable for sterilization between uses.

Aspect 165: The adjustable sheath apparatus of claim 43, wherein the fluid channel includes a valve mechanism to control the flow rate of irrigation fluid.

Aspect 166: The adjustable sheath apparatus of claim 43, wherein the sheath body is designed to minimize optical distortion when used in conjunction with the endoscope.

Aspect 167: The adjustable sheath apparatus of claim 43, wherein the sheath body includes an ergonomic profile to facilitate handling and positioning during the endoscopic procedure.

Aspect 168: The adjustable sheath apparatus of claim 43, wherein the sheath body is designed to reduce glare and reflections that may interfere with the endoscopic imaging.

Aspect 169: The adjustable sheath apparatus of claim 43, wherein the sheath body includes a textured exterior surface to improve grip and control during manipulation.

Aspect 170: The adjustable sheath apparatus of claim 43, wherein the sheath body is designed to be disposable for single-use applications.

Aspect 171: The adjustable sheath apparatus of claim 43, wherein the sheath body includes an integrated channel for passing additional instruments alongside the endoscope.

Aspect 172: A mechanical foot pedal assembly for controlling suction and irrigation in a surgical hand piece apparatus during endoscopic procedures, comprising: a base structure configured to support a user's foot actions; a first pedal pivotally attached to the base structure and associated with a first spring-loaded pinch valve for controlling irrigation fluid flow; a second pedal pivotally attached to the base structure and associated with a second spring-loaded pinch valve for controlling suction flow; a set of color-coded indicators on the first and second pedals for distinguishing between the irrigation and suction controls; and a tubing retention system integrated into the base structure for securing the flexible suction and irrigation tubing from the apparatus.

Aspect 173: The mechanical foot pedal assembly of claim 43, wherein the base structure includes a non-skid surface to prevent movement during operation.

Aspect 174: The mechanical foot pedal assembly of claim 43, wherein the first and second spring-loaded pinch valves are configured to provide continuous and uninterrupted fluid flow when actuated.

Aspect 175: The mechanical foot pedal assembly of claim 43, wherein the first and second pedals are configured to return to a default position that closes the pinch valves when not engaged by the user.

Aspect 176: The mechanical foot pedal assembly of claim 43, wherein the color-coded indicators comprise visually distinct colors that correspond to universal color standards for medical irrigation and suction.

Aspect 177: The mechanical foot pedal assembly of claim 43, wherein the tubing retention system includes adjustable clamps to accommodate various tubing diameters.

Aspect 178: The mechanical foot pedal assembly of claim 43, further comprising a heavy-duty construction of the base structure to provide stability and durability during repeated use.

Aspect 179: The mechanical foot pedal assembly of claim 43, wherein the spring-loaded pinch valves are replaceable for maintenance and longevity of the assembly.

Aspect 180: The mechanical foot pedal assembly of claim 43, wherein the first and second pedals are ergonomically designed to minimize foot strain during extended surgical procedures.

Aspect 181: The mechanical foot pedal assembly of claim 43, wherein the base structure includes a carrying handle for portability.

Aspect 182: The mechanical foot pedal assembly of claim 43, wherein the base structure is constructed from a material resistant to common sterilization processes.

Aspect 183: The mechanical foot pedal assembly of claim 43, wherein the first and second pedals include textured surfaces to enhance grip and control.

Aspect 184: The mechanical foot pedal assembly of claim 43, wherein the base structure is designed to facilitate easy cleaning and disinfection.

Aspect 185: The mechanical foot pedal assembly of claim 43, wherein the tubing retention system is designed to prevent accidental dislodgement of the tubing during use.

Aspect 186: The mechanical foot pedal assembly of claim 43, wherein the assembly is compatible with a range of surgical hand piece apparatuses from various manufacturers.

Aspect 187: The mechanical foot pedal assembly of claim 43, wherein the assembly includes a locking mechanism to maintain the selected flow rate of irrigation fluid or suction.

Aspect 188: The mechanical foot pedal assembly of claim 43, wherein the assembly is designed to operate without the need for electrical power.

Aspect 189: The mechanical foot pedal assembly of claim 43, wherein the assembly includes a feedback mechanism to provide tactile or auditory confirmation of valve actuation.

Aspect 190: The mechanical foot pedal assembly of claim 43, wherein the assembly is designed to be operated by either foot of the user.

Aspect 191: The mechanical foot pedal assembly of claim 43, wherein the assembly includes a modular design for easy replacement of individual components.

Aspect 192: A method for providing continuous suction and irrigation during an endoscopic procedure, comprising: providing a surgical hand piece apparatus with an adjustable sheath, a combined suction and irrigation port, and a mechanical foot pedal assembly; inserting an endoscope into the adjustable sheath of the surgical hand piece apparatus; connecting the combined suction and irrigation port to a source of irrigation fluid and a suction device; actuating a first pedal of the mechanical foot pedal assembly to control the flow of irrigation fluid through the combined suction and irrigation port and the adjustable sheath to the endoscope; and actuating a second pedal of the mechanical foot pedal assembly to control the suction through the combined suction and irrigation port and the adjustable sheath from the endoscope.

Aspect 193: The method of claim 43, wherein adjusting the length of the adjustable sheath to accommodate the endoscope of varying lengths prior to the inserting step.

Aspect 194: The method of claim 43, wherein the actuating steps for the first and second pedals are performed using color-coded pedals for easy identification of the irrigation and suction controls.

Aspect 195: The method of claim 43, further comprising supporting the surgical hand piece apparatus on the thenar eminence of a user's hand during the inserting step.

Aspect 196: The method of claim 43, wherein the providing step includes selecting a surgical hand piece apparatus with an ergonomic hand grip portion designed to reduce carpal tunnel pressure and fatigue.

Aspect 197: The method of claim 43, further comprising maintaining the mechanical foot pedal assembly in a default position that closes the spring-loaded pinch valves when the pedals are not engaged.

Aspect 198: The method of claim 43, wherein the connecting step includes using a single Luer lock or barbed fitting to establish the connection to the source of irrigation fluid and the suction device.

Aspect 199: These method aspects outline the steps for using the surgical hand piece apparatus with the novel features disclosed in the invention, such as the adjustable sheath, combined suction and irrigation port, and mechanical foot pedal assembly. Each dependent aspect adds specific actions or features that refine the method, potentially increasing the scope of patent protection for the use of the apparatus in endoscopic procedures.

Aspect 200: A method for maintaining visibility and cleanliness of an endoscopic lens during a surgical procedure, comprising: providing a surgical hand piece with an integrated adjustable sheath and a combined suction and irrigation port; inserting an endoscope through the adjustable sheath; connecting a source of irrigation fluid and a suction device to the combined suction and irrigation port; actuating a mechanical foot pedal assembly to control the flow of irrigation fluid to the endoscope; and actuating the mechanical foot pedal assembly to control suction from the operative field through the endoscope.

Aspect 201: The method of claim 43, wherein the adjustable sheath is configured to accommodate endoscopes of various diameters.

Aspect 202: The method of claim 43, wherein the irrigation fluid includes an antibiotic solution.

Aspect 203: The method of claim 43, wherein the irrigation fluid is warmed to facilitate paradoxical vasoconstriction during the surgical procedure.

Aspect 204: The method of claim 43, wherein the mechanical foot pedal assembly includes spring-loaded pinch valves for controlling the flow of irrigation fluid and suction.

Aspect 205: The method of claim 43, wherein the mechanical foot pedal assembly is non-electronic.

Aspect 206: The method of claim 43, wherein the surgical hand piece includes an ergonomic design to reduce fatigue during the surgical procedure.

Aspect 207: The method of claim 43, further comprising adjusting the effective length of the adjustable sheath to match the length of the endoscope.

Aspect 208: The method of claim 43, wherein the combined suction and irrigation port utilizes a single Luer lock or barbed fitting for both suction and irrigation.

Aspect 209: The method of claim 43, wherein the actuating step for controlling irrigation fluid is performed independently from the actuating step for controlling suction.

Aspect 210: The method of claim 43, further comprising cleaning the lens of the endoscope with the irrigation fluid without removing the endoscope from the operative field.

Aspect 211: The method of claim 43, wherein the suction is used to evacuate blood and debris from the operative field.

Aspect 212: The method of claim 43, wherein the mechanical foot pedal assembly includes color-coded pedals for distinguishing between irrigation and suction controls.

Aspect 213: The method of claim 43, wherein the surgical hand piece is supported on the thenar eminence of the user's hand during the procedure.

Aspect 214: The method of claim 43, wherein the surgical hand piece includes a soft grip material over a hard molded housing for comfort.

Aspect 215: The method of claim 43, wherein the surgical hand piece is optimized for use during sinuplasty procedures.

Aspect 216: The method of claim 43, wherein the mechanical foot pedal assembly provides continuous control over the flow of fluids.

Aspect 217: The method of claim 43, wherein the surgical hand piece accommodates multiple scopes from various manufacturers.

Aspect 218: The method of claim 43, wherein the surgical hand piece includes a sheath with an internal sealing o-ring to maintain a fluid-tight seal around the endoscope.

Aspect 219: The method of claim 43, wherein the surgical hand piece includes a flow valve assembly operative to direct fluid flow through the adjustable sheath.

Aspect 220: The method of claim 43, wherein the surgical hand piece is designed to provide carpal tunnel fatigue relief.

Aspect 221: The method of claim 43, wherein the mechanical foot pedal assembly is configured to operate the spring-loaded pinch valves independently for suction and irrigation.

Aspect 222: The method of claim 43, wherein the surgical hand piece and the adjustable sheath are configured to allow for quick disassembly for cleaning and sterilization.

Aspect 223: The method of claim 43, wherein the surgical hand piece includes a rigid structure with a soft rubber overmold.

Aspect 224: The method of claim 43, wherein the surgical hand piece is universally compatible with endoscopes from multiple manufacturers.

Aspect 225: The method of claim 43, wherein the surgical hand piece includes a thenar rest to allow support of the hand piece on the thumb web.

Aspect 226: The method of claim 43, wherein the surgical hand piece includes a soft grip cover that is overmolded.

Aspect 227: The method of claim 43, wherein the surgical hand piece includes an adjustable length sheath with a designed Luer lock.

Aspect 228: The method of claim 43, wherein the surgical hand piece includes a suction/irrigation combined port attached to the hand piece.

Aspect 229: The method of claim 43, wherein the surgical hand piece includes a mechanical foot pedal with pedals for suction and irrigation, and the two valves are color-coded for easy application by the operating room staff.

Aspect 230: A method for facilitating an endoscopic procedure with an adjustable sheath, comprising: providing a surgical hand piece with an integrated adjustable sheath capable of accommodating various endoscope lengths; inserting an endoscope into the adjustable sheath until a desired length is achieved; securing the endoscope within the adjustable sheath using a securement feature; directing irrigation fluid through a fluid channel in the adjustable sheath to the distal end of the endoscope; and facilitating suction from the proximal end of the endoscope through the fluid channel in the adjustable sheath.

Aspect 231: The method of claim 43, wherein the step of providing a surgical hand piece includes selecting a sheath body constructed from a biocompatible and corrosion-resistant material.

Aspect 232: The method of claim 43, further comprising adjusting the effective length of the adjustable sheath using a sliding collar mechanism integrated into the sheath body.

Aspect 233: The method of claim 43, wherein the securement feature comprises a Luer lock mechanism that maintains a fluid-tight connection with the endoscope.

Aspect 234: The method of claim 43, further comprising controlling the flow rate of irrigation fluid through a valve mechanism within the fluid channel.

Aspect 235: The method of claim 43, wherein the step of directing irrigation fluid includes using the fluid channel to maintain separation between the irrigation fluid and the suctioned materials.

Aspect 236: The method of claim 43, further comprising detaching the adjustable sheath from the surgical hand piece using a quick-release mechanism after completing the endoscopic procedure.

Aspect 237: The method of claim 43, wherein the step of inserting the endoscope includes aligning graduated markings on the adjustable sheath with a reference point on the endoscope to determine the adjusted length.

Aspect 238: The method of claim 43, further comprising sterilizing the adjustable sheath by autoclaving between uses.

Aspect 239: The method of claim 43, wherein the step of facilitating suction includes evacuating blood and debris from the operative field through the fluid channel.

Aspect 240: The method of claim 43, further comprising passing additional instruments alongside the endoscope through an integrated channel in the adjustable sheath.

Aspect 241: The method of claim 43, wherein the step of providing a surgical hand piece includes selecting an adjustable sheath with an ergonomic profile for ease of handling.

Aspect 242: The method of claim 43, further comprising minimizing optical distortion during the endoscopic procedure by using an adjustable sheath designed to reduce glare and reflections.

Aspect 243: The method of claim 43, wherein the step of securing the endoscope includes engaging an internal sealing element within the adjustable sheath to enhance the fluid-tight connection.

Aspect 244: The method of claim 43, further comprising disposing of the adjustable sheath after a single use to maintain procedural sterility.

Aspect 245: These method aspects focus on the procedural steps enabled by the invention, detailing the use of the surgical hand piece during an endoscopic procedure. Each dependent aspect adds specific actions or features that refine the method, potentially increasing the scope of patent protection for the use of the apparatus in endoscopic procedures.

Aspect 246: A method for controlling fluid flow during an endoscopic procedure, comprising: providing a surgical hand piece with an integrated adjustable sheath and a combined suction and irrigation port; adjusting a valve mechanism within the adjustable sheath to regulate irrigation fluid flow to the endoscope; actuating a control interface on the surgical hand piece to modify the suction force through the endoscope; and maintaining continuous fluid flow and suction during the endoscopic procedure through user manipulation of the control interface.

Aspect 247: The method of claim 43, wherein adjusting the valve mechanism includes actuating a spring-loaded pinch valve for the irrigation pathway.

Aspect 248: The method of claim 43, further comprising monitoring the fluid flow through a transparent section of the adjustable sheath.

Aspect 249: The method of claim 43, wherein the step of providing a surgical hand piece includes selecting a hand piece with a single connection point for both irrigation and suction.

Aspect 250: The method of claim 43, wherein actuating the control interface involves using an ergonomic actuator designed to minimize user fatigue.

Aspect 251: The method of claim 43, further comprising replacing the valve mechanism as part of maintenance of the surgical hand piece.

Aspect 252: The method of claim 43, wherein maintaining continuous fluid flow includes preventing backflow and cross-contamination by utilizing a combined suction and irrigation port with anti-backflow features.

Aspect 253: The method of claim 43, further comprising sterilizing the adjustable sheath and valve mechanism using standard medical sterilization processes.

Aspect 254: A method for capturing and transmitting endoscopic images, comprising: capturing visual data from an operative field using an electronic image sensor within an endoscope; processing the visual data into electronic image signals with a data processing unit; transmitting the electronic image signals to an external processing system via a transmission module; and powering the electronic image sensor and data processing unit with a power supply mechanism.

Aspect 255: The method of claim 43, further comprising enhancing the electronic image signals using an image signal processor to improve image quality.

Aspect 256: The method of claim 43, wherein transmitting the electronic image signals includes utilizing a wireless communication interface to send data to a remote display.

Aspect 257: The method of claim 43, further comprising managing power consumption by monitoring the charge level of a rechargeable battery within the endoscope.

Aspect 258: The method of claim 43, wherein capturing visual data involves using a high-resolution image sensor to obtain detailed images for clinical analysis.

Aspect 259: The method of claim 43, further comprising adhering to medical image storage and communication protocols during the transmission of electronic image signals.

Aspect 260: The method of claim 43, wherein processing the visual data includes applying real-time image stabilization to maintain image clarity despite movement.

Aspect 261: The method of claim 43, further comprising alerting the user of low battery status to ensure uninterrupted power supply during the endoscopic procedure.

Aspect 262: The method of claim 43, wherein transmitting the electronic image signals involves high-bandwidth data transfer to provide real-time imaging to the user.

Aspect 263: The method of claim 43, wherein capturing visual data includes adjusting the sensitivity of the image sensor to accommodate varying light conditions within the operative field.

Aspect 264: A method for enhancing visualization during an endoscopic procedure, comprising: inserting an endoscope with an integrated camera into an adjustable sheath; delivering an irrigation fluid through the adjustable sheath to maintain cleanliness of the endoscope lens; capturing images of the operative field with the integrated camera; and transmitting the captured images to an external display via a connection interface.

Aspect 266: The method of claim 43, further comprising adjusting the flow of the irrigation fluid using a valve mechanism to optimize lens clarity.

Aspect 267: The method of claim 43, wherein transmitting the captured images includes utilizing a high-definition video output for enhanced image resolution.

Aspect 268: The method of claim 43, further comprising utilizing the zoom function of the integrated camera to focus on specific areas of interest within the operative field.

Aspect 269: The method of claim 43, wherein delivering the irrigation fluid includes using a steady stream to prevent dynamic interference with the imaging process.

Aspect 270: The method of claim 43, further comprising processing the captured images in real-time to enhance visualization of the operative field.

Aspect 271: The method of claim 43, wherein the step of inserting the endoscope into the adjustable sheath includes aligning the endoscope lens with a transparent section of the sheath for direct visual inspection.

Aspect 272: The method of claim 43, further comprising sterilizing the adjustable sheath and fluid control system using standard medical sterilization processes without affecting the integrated camera.

Aspect 273: A method for customizing a surgical hand piece for endoscopic procedures, comprising: selecting an ergonomic grip module from a set of interchangeable modules based on a user's hand size and preference; attaching the selected ergonomic grip module to a main body of the surgical hand piece using a quick-connect mechanism; choosing a fluid control module from a set of interchangeable modules to match the procedural irrigation and suction needs; connecting the chosen fluid control module to the main body; and securing a detachable adjustable sheath to the main body to accommodate the endoscope being used.

Aspect 275: The method of claim 43, further comprising swapping the ergonomic grip module with another from the set to adapt to different users or ergonomic requirements.

Aspect 276: The method of claim 43, wherein choosing a fluid control module includes selecting a module with automated flow regulation for precision control.

Aspect 277: The method of claim 43, further comprising utilizing the locking collar of the quick-connect mechanism to ensure a secure yet easily reversible attachment.

Aspect 278: The method of claim 43, wherein securing the detachable adjustable sheath includes selecting a sheath with a specific diameter from a range of sheath sizes.

Aspect 279: The method of claim 43, further comprising organizing and storing the interchangeable modules in a designated storage case to maintain readiness for various procedures.

Aspect 280: The method of claim 43, wherein attaching the ergonomic grip module involves aligning electronic connectors for integration with endoscope imaging systems.

Aspect 281: The method of claim 43, further comprising conducting an ergonomic assessment to determine the most suitable ergonomic grip module for the user.

Aspect 282: The method of claim 43, wherein choosing a fluid control module is facilitated by identifying the color-coded modules corresponding to the procedural requirements.

Aspect 283: The method of claim 43, further comprising sterilizing the main body and detachable components using approved disinfection and sterilization techniques without disassembly.

Aspect 284: A method for sterilizing an endoscopic apparatus, comprising: disassembling the endoscopic apparatus into components suitable for sterilization; subjecting the components to a high-temperature autoclave sterilization process; performing chemical disinfection on components sensitive to high temperatures; reassembling the sterilized components into a functional endoscopic apparatus; and verifying the sterilization efficacy through visual indicators on the components.

Aspect 285: The method of claim 43, wherein disassembling the endoscopic apparatus includes separating silicone or elastomer-based fluid control system components.

Aspect 286: The method of claim 43, further comprising using an antimicrobial coating on the detachable sheath to enhance the effects of the autoclave sterilization process.

Aspect 287: The method of claim 43, wherein performing chemical disinfection involves immersing the components in a glutaraldehyde-based solution.

Aspect 288: The method of claim 43, further comprising encapsulating electronic components in a protective housing to enable chemical disinfection without damage.

Aspect 289: The method of claim 43, wherein reassembling the sterilized components includes attaching a camera module that has been separately sterilized.

Aspect 290: The method of claim 43, further comprising conducting repeated sterilization cycles and inspecting for material integrity to ensure long-term usability.

Aspect 291: The method of claim 43, wherein disassembling the fluid control system for sterilization includes following a standardized protocol to maintain component functionality.

Aspect 292: The method of claim 43, further comprising utilizing color-changing indicators to provide visual confirmation of successful sterilization.

Aspect 293: The method of claim 43, wherein verifying sterilization efficacy includes testing for the presence of biological indicators post-sterilization.

Aspect 294: A method for managing irrigation and suction during endoscopic procedures, comprising: providing an endoscopic apparatus with an integrated fluid control system; delivering irrigation fluid to the operative field through a dedicated irrigation channel; removing fluids from the operative field using a dedicated suction channel; independently controlling the flow rates of irrigation and suction via a valve assembly; and collecting the removed fluids in a waste collection container.

Aspect 295: The method of claim 43, further comprising adjusting the valve assembly to modify the flow rates in response to the procedural requirements.

Aspect 296: The method of claim 43, wherein delivering irrigation fluid includes using a sterile fluid reservoir to prevent contamination.

Aspect 297: The method of claim 43, further comprising filtering the suctioned fluids to separate debris before collection in the waste container.

Aspect 298: The method of claim 43, wherein managing the pressure of the irrigation and suction involves a regulation system to maintain consistent operation.

Aspect 299: The method of claim 43, further comprising monitoring the fluid levels and flow rates through a user interface on the surgical hand piece.

Aspect 300: The method of claim 43, wherein performing simultaneous irrigation and suction includes preventing cross-contamination between the channels.

Aspect 301: The method of claim 43, further comprising operating the valve assembly with a foot pedal to maintain a sterile field and free the user's hands for other tasks.

Aspect 302: The method of claim 43, wherein collecting the removed fluids involves detaching the waste collection container for disposal and replacing it with an empty container as needed.

Aspect 303: The method of claim 43, further comprising maintaining the dual-channel tubing system to ensure unobstructed flow and effective removal of fluids throughout the procedure.

Aspect 304: A method for enhancing ergonomic usability of an endoscopic apparatus, comprising: providing a surgical hand piece with a design based on human factors engineering; adjusting the weight distribution within the hand piece to achieve ergonomic balance; customizing the grip texture and support features to fit the user's hand; positioning the user interface for easy access to apparatus controls; and utilizing ergonomic visual indicators to guide the user in making adjustments for optimal comfort and usability.

Aspect 305: The method of claim 43, further comprising adding a padded overlay to the grip for prolonged procedural comfort.

Aspect 306: The method of claim 43, wherein adjusting the weight distribution includes shifting internal components or adding counterweights.

Aspect 307: The method of claim 43, further comprising selecting a grip texture from a variety of non-slip materials to suit the user's preference.

Aspect 308: The method of claim 43, wherein customizing the grip involves modifying thumb rests and finger grooves for individual hand ergonomics.

Aspect 309: The method of claim 43, further comprising integrating touch-sensitive controls into the user interface to minimize the force required for operation.

Aspect 310: The method of claim 43, further comprising attaching a detachable wrist support to the hand piece to support a neutral hand and wrist posture.

Aspect 311: The method of claim 43, wherein enhancing ergonomic usability includes designing the hand piece to distribute pressure evenly and reduce the risk of repetitive strain injuries.

Aspect 312: The method of claim 43, further comprising insulating the grip areas to protect the user's hands from temperature extremes during procedures.

Aspect 313: The method of claim 43, wherein utilizing ergonomic visual indicators involves providing feedback on the hand piece to ensure the user maintains an ergonomically sound grip throughout the procedure.

Aspect 314: A method for performing a surgical procedure using an endoscopic apparatus with integrated fluid control and ergonomic design, comprising: positioning the surgical hand piece of the endoscopic apparatus to access the operative field; activating the fluid control system to deliver irrigation fluid to the operative field and suction fluids from the operative field; manipulating the endoscope to visualize the surgical site through a remote visualization unit; adjusting the ergonomic features of the hand piece to conform to the user's hand for comfortable operation; and operating the endoscopic apparatus controls to perform the surgical procedure while maintaining ergonomic posture.

Aspect 315: The method of claim 43, further comprising adjusting the flow rate of irrigation and suction using the fluid control system to match the procedural requirements.

Aspect 316: The method of claim 43, wherein positioning the surgical hand piece includes utilizing the weight distribution system to minimize user fatigue.

Aspect 317: The method of claim 43, further comprising utilizing wireless communication capabilities of the endoscopic apparatus to transmit visual data to the remote visualization unit.

Aspect 318: The method of claim 43, wherein adjusting the ergonomic features includes configuring the contoured grip and support features to reduce pressure points on the user's hand.

Aspect 319: The method of claim 43, further comprising using touch-sensitive controls on the hand piece to adjust endoscopic settings without significant hand movement.

Aspect 320: The method of claim 43, wherein manipulating the endoscope includes using a joystick or similar control mechanism integrated into the hand piece for precise movements.

Aspect 321: The method of claim 43, further comprising monitoring the fluid levels in the reservoir and waste collection container through indicators on the hand piece.

Aspect 322: The method of claim 43, wherein operating the endoscopic apparatus controls includes using a foot pedal mechanism to maintain a sterile field.

Aspect 323: The method of claim 43, further comprising sterilizing the endoscopic apparatus before the surgical procedure using compatible sterilization processes.

Aspect 324: The method of claim 43, wherein activating the fluid control system includes providing simultaneous irrigation and suction without cross-contamination.

Aspect 325: The method of claim 43, further comprising employing a pressure regulation system within the fluid control system to maintain consistent flow during the procedure.

Aspect 326: The method of claim 43, wherein adjusting the ergonomic features includes selecting a grip texture that enhances grip stability during the surgical procedure.

Aspect 327: The method of claim 43, further comprising utilizing a detachable wrist support attached to the hand piece to maintain the user's hand in a neutral position.

Aspect 328: The method of claim 43, wherein operating the endoscopic apparatus controls includes utilizing haptic feedback to confirm the execution of control inputs.

Aspect 329: The method of claim 43, further comprising conducting the surgical procedure with minimal hand movement by positioning the user interface for intuitive access.

Aspect 330: The method of claim 43, wherein manipulating the endoscope includes utilizing a synchronization module to ensure a stable wireless connection for real-time visualization.

Aspect 331: The method of claim 43, further comprising adjusting the valve assembly to alternate between irrigation and suction as dictated by the surgical procedure.

Aspect 332: The method of claim 43, wherein adjusting the ergonomic features includes using visual indicators on the hand piece to guide the user to an optimal grip configuration.

Aspect 333: The method of claim 43, further comprising engaging a locking mechanism on the hand piece to secure the endoscope during the surgical procedure.

Aspect 334: A method for configuring a modular endoscopic apparatus for specific surgical procedures, comprising: selecting an endoscope module from a set of interchangeable modules based on the surgical procedure; attaching the selected endoscope module to a base surgical hand piece using a universal connector; customizing a fluid control system with modular components to meet the irrigation and suction needs of the procedure; choosing an ergonomic grip from a selection of grips to fit the user's hand and procedure requirements; and programming a user interface module with control settings specific to the surgical procedure.

Aspect 335: The method of claim 43, further comprising utilizing a quick-release mechanism on the universal connector for efficient tool exchanges during surgery.

Aspect 336: The method of claim 43, wherein selecting an endoscope module includes considering the operative field size and required visualization depth.

Aspect 337: The method of claim 43, further comprising assembling the fluid control system with selected valves and pumps to achieve desired flow rates.

Aspect 338: The method of claim 43, wherein choosing an ergonomic grip involves assessing the duration of the procedure and the user's grip strength.

Aspect 339: The method of claim 43, further comprising programming the user interface module with a touch screen to create a custom control layout.

Aspect 340: The method of claim 43, further comprising selecting a sheath from a variety of sizes to match the chosen endoscope module.

Aspect 341: The method of claim 43, further comprising interchanging power supply modules to ensure adequate battery life for the duration of the procedure.

Aspect 342: The method of claim 43, wherein customizing the fluid control system includes matching color-coded modules to the corresponding procedure type.

Aspect 343: The method of claim 43, further comprising wirelessly updating the user interface module with the latest control settings and software prior to the procedure.

Aspect 344: A method for integrating an endoscopic apparatus with various surgical environments and equipment, comprising: connecting the apparatus to a surgical monitor using a universal interface; adapting the power supply of the apparatus to match the surgical environment's electrical standards; calibrating the apparatus based on readings from surgical environment sensors; synchronizing the apparatus with hospital information systems for seamless data exchange; and utilizing a sterilizable docking station for charging and data synchronization between surgeries.

Aspect 345: The method of claim 43, further comprising selecting the appropriate video output format for compatibility with the surgical monitor's display capabilities.

Aspect 346: The method of claim 43, wherein adapting the power supply includes automatically adjusting to the voltage and frequency of the local power grid.

Aspect 347: The method of claim 43, further comprising adjusting the apparatus settings in response to changes in ambient surgical conditions detected by the sensors.

Aspect 348: The method of claim 43, wherein synchronizing the apparatus involves establishing a secure wireless connection with the hospital's network.

Aspect 349: The method of claim 43, further comprising tracking the apparatus's usage and sterilization cycles using RFID or barcode technology integrated into the docking station.

Aspect 350: The method of claim 43, further comprising selecting and attaching modular accessories from the accessory kit to prepare the apparatus for specific surgical procedures.

Aspect 351: The method of claim 43, further comprising updating the apparatus's firmware and software remotely through the communication module.

Aspect 352: The method of claim 43, further comprising interfacing the apparatus with a robotic surgical system to enhance precision and control during surgery.

Aspect 353: The method of claim 43, further comprising authenticating authorized users through the docking station before accessing the apparatus.

Aspect 354: A method for remote visualization and control during endoscopic procedures, comprising: wirelessly transmitting visual data from an endoscope to a remote visualization unit; displaying the received visual data on the remote visualization unit; remotely adjusting endoscopic functions via a control interface on the endoscope; and synchronizing the endoscope and the remote visualization unit to maintain a secure wireless connection.

Aspect 355: The method of claim 43, further comprising operating the integrated wireless transmitter on a dedicated medical frequency band to ensure reliable communication.

Aspect 356: The method of claim 43, wherein displaying the received visual data includes utilizing a high-definition display for enhanced image clarity.

Aspect 357: The method of claim 43, further comprising remotely controlling endoscopic settings such as zoom, focus, and image capture through the control interface.

Aspect 358: The method of claim 43, wherein synchronizing the endoscope and the remote visualization unit involves implementing encryption protocols to protect patient data.

Aspect 359: The method of claim 43, further comprising receiving control signals at the endoscope from the remote visualization unit to facilitate bidirectional communication.

Aspect 360: The method of claim 43, wherein the remote visualization unit's touch-screen capabilities are used to adjust settings and navigate through images and videos.

Aspect 361: The method of claim 43, further comprising charging the endoscope's battery wirelessly to minimize downtime between procedures.

Aspect 362: The method of claim 43, wherein synchronizing the endoscope and the remote visualization unit includes supporting various wireless communication standards for interoperability.

Aspect 363: The method of claim 43, further comprising providing haptic feedback on the control interface to indicate successful reception of remote commands.

Both the foregoing overview and the following detailed description provide examples and are explanatory only. Accordingly, the foregoing overview and the following detailed description should not be considered to be restrictive. Further, features or variations may be provided in addition to those set forth herein. For example, embodiments may be directed to various feature combinations and sub-combinations described in the detailed description.

The embodiments described herein offer a scope device that is comfortable for a user to hold. The device may provide continuous irrigation and suction to the operational space while accommodating a variety of sheaths. Both the foregoing overview and the following detailed description provide examples and are explanatory only.

Accordingly, the foregoing overview and the following detailed description should not be considered to be restrictive. Further, features or variations may be provided in addition to those set forth herein. For example, embodiments may be directed to various feature combinations and sub-combinations described in the detailed description.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

1. An apparatus for endoscopic procedures, comprising:
a body portion configured to receive and retain an endoscope;
an adjustable sheath coupled to the body portion, wherein the adjustable sheath is configured to accommodate endoscopes of varying lengths;
a combined suction and irrigation port integrated into the body portion;
a mechanical foot pedal assembly communicatively connected to the combined suction and irrigation port, the mechanical foot pedal assembly including spring-loaded pinch valves for controlling fluid flow; and
an ergonomic hand grip portion formed on the body portion, the ergonomic hand grip portion including a soft overmolded material designed to reduce carpal tunnel pressure and fatigue during use.

2. (canceled)

3. The apparatus of claim 1, wherein the adjustable sheath includes a Luer lock mechanism for securing the endoscope.

4. The apparatus of claim 1, wherein the combined suction and irrigation port comprises a single Luer lock or barbed fitting.

5. The apparatus of claim 1, wherein the mechanical foot pedal assembly is non-electronic.

6. The apparatus of claim 1, wherein the spring-loaded pinch valves are color-coded for easy identification of suction and irrigation controls.

7. The apparatus of claim 1, wherein the ergonomic hand grip portion is designed to support the thenar eminence of a user's hand.

8. The apparatus of claim 1, wherein the body portion further comprises a rigid structure with a soft rubber overmold.

9. The apparatus of claim 1, wherein the adjustable sheath is configured to provide a fluid flow path for both irrigation and suction fluids.

10. (canceled)

11. The apparatus of claim 9, wherein the adjustable sheath is configured to translate relative to the body portion to accommodate different endoscope models.

12. (canceled)

13. The apparatus of claim 6, wherein the mechanical foot pedal assembly is configured to return to a default position that closes the spring-loaded pinch valves when not engaged.

14. The apparatus of claim 6, wherein the combined suction and irrigation port is configured to allow continuous flow of fluids without interruption.

15.-42. (canceled)

43. A method for providing continuous suction and irrigation during an endoscopic procedure, comprising:
providing a surgical hand piece apparatus with an adjustable sheath, a combined suction and irrigation port, and a mechanical foot pedal assembly;
inserting an endoscope into the adjustable sheath of the surgical hand piece apparatus; connecting the combined suction and irrigation port to a source of irrigation fluid and a suction device;
actuating a first pedal of the mechanical foot pedal assembly to control the flow of irrigation fluid through the combined suction and irrigation port and the adjustable sheath to the endoscope; and
actuating a second pedal of the mechanical foot pedal assembly to control the suction through the combined suction and irrigation port and the adjustable sheath from the endoscope.

44. The method of claim 43, wherein adjusting the length of the adjustable sheath to accommodate the endoscope of varying lengths prior to the inserting step.

45. The method of claim 43, wherein the actuating steps for the first and second pedals are performed using color-coded pedals for easy identification of the irrigation and suction controls.

46. The method of claim 43, further comprising supporting the surgical hand piece apparatus on the thenar eminence of a user's hand during the inserting step.

47. The method of claim 43, wherein the providing step includes selecting a surgical hand piece apparatus with an ergonomic hand grip portion designed to reduce carpal tunnel pressure and fatigue.

48. The method of claim 43, further comprising maintaining the mechanical foot pedal assembly in a default position that closes the spring-loaded pinch valves when the pedals are not engaged.

49. The method of claim 43, wherein the connecting step includes using a single Luer lock or barbed fitting to establish the connection to the source of irrigation fluid and the suction device.

50. An apparatus for endoscopic procedures, comprising:
a body configured to accommodate an endoscope;
an ergonomic grip portion designed to conform to the contours of a user's hand;
an integrated adjustable sheath for encasing a portion of the endoscope and allowing length adjustment;
a combined suction and irrigation port connected to the body for providing fluid flow and suction through the endoscope; and
a thenar rest positioned to support the hand piece on the user's thenar eminence during use;
wherein the adjustable sheath includes a Luer lock mechanism for securing the endoscope;
wherein the combined suction and irrigation port comprises a single Luer lock or barbed fitting; and
wherein the adjustable sheath is configured to provide a fluid flow path for both irrigation and suction fluids and to translate relative to the body portion to accommodate different endoscope models.

51. The apparatus of claim 50 further comprising spring-loaded pinch valves which are color-coded for identification of suction and irrigation controls.
